(19) United States
(12) Patent Application Publication
(10) Pub. No.: US 2026/0272876 A1
(43) Pub. Date: Sep. 17, 2026

(54) METHODS FOR DEVELOPING COMPANION SUPPLEMENTS FOR DOGS, RELATED COMPOSITIONS, AND SYSTEMS

(71) Applicant: Life's Abundance, Jupiter, FL (US)

(72) Inventor: Lester Thornhill, Jupiter, FL (US)

(73) Assignee: Life's Abundance, Jupiter, FL (US)

(21) Appl. No.: 19/424,844

(22) Filed: Dec. 18, 2025

Related U.S. Application Data
(63) Continuation-in-part of application No. 19/080,493, filed on Mar. 14, 2025, now abandoned.

(51) Int. Cl. A61K 31/375; A23K 10/20; A23K 10/30; A23K 20/142; A23K 20/147; A23K 20/158; A23K 20/174; A23K 20/24; A23K 20/26; A23K 50/42; A23K 50/60; A61K 9/00; A61K 31/10; A61K 31/12; A61K 31/122; A61K 31/14; A61K 31/198; A61K 31/201; A61K 31/202; A61K 31/205; A61K 31/355; A61K 31/405; A61K 31/4188; A61K 31/7008; A61K 31/702; A61K 31/7052; A61K 31/715; A61K 31/716; A61K 31/733; A61K 31/737; A61K 33/24; A61K 33/30; A61K 33/32; A61K 35/00; A61K 35/741; A61K 36/03; A61K 36/235; A61K 36/28; A61K 36/324; A61K 36/54; A61K 36/67; A61K 36/81; A61K 36/82; A61K 36/9068; A61K 38/39; A61K 38/46; A61K 38/47; A61K 38/48

(52) U.S. Cl. CPC A61K 31/375; A23K 10/20; A23K 10/30; A23K 20/142; A23K 20/147; A23K 20/158; A23K 20/174; A23K 20/24; A23K 20/26; A23K 50/42; A23K 50/60; A61K 9/0056; A61K 31/10; A61K 31/12; A61K 31/122; A61K 31/14; A61K 31/198; A61K 31/201; A61K 31/202; A61K 31/205; A61K 31/355; A61K 31/405; A61K 31/4188; A61K 31/7008; A61K 31/702; A61K 31/7052; A61K 31/715; A61K 31/716; A61K 31/733; A61K 31/737; A61K 33/24; A61K 33/30; A61K 33/32; A61K 35/741; A61K 36/03; A61K 36/235; A61K 36/28; A61K 36/324; A61K 36/54; A61K 36/67; A61K 36/81; A61K 36/82; A61K 36/9068; A61K 38/39; A61K 38/465; A61K 38/47; A61K 38/4873; C12Y 304/22033; C12Y 304/22039; A61K 2035/115

(57) ABSTRACT

Methods for developing a well-rounded and nutritionally optimal nutritional plan for a dog are provided herein. The method comprises choosing a base food format, wherein the base food format comprises base food ingredients; determining post-cook characteristics of the base food format, wherein the post-cook characteristics are selected from the group consisting of nutrient levels, temporal nutrient degradation modeling across an expected shelf-life of the base food format, bioavailability, digestibility, nutrient antagonisms, chemical form, oxidation losses over a shelf-life of the base food format, stability over a shelf-life of the base food format, breed-specific risk factors, and combinations thereof; preparing a companion supplement based, at least in part, on the post-cook characteristics of the base food format, wherein the companion supplement comprises companion supplement ingredients, and wherein the companion supplement ingredients are selected from one of a limiting amino acid that is not present in the base food format, one or more enzymes calibrated to a protein:fat:carbohydrate ratio of the base food format, and combinations thereof; determining post-production characteristics of the companion supplement; and combining nutrient values of the base food format and the companion supplement to ensure safety and efficacy, and wherein the companion supplement and the base food format, when administered together, are sufficiently designed to provide the dog with a nutritionally optimal feeding system for the dog based on the dog's stage of life and nutritional requirements.

### CROSS-REFERENCE TO RELATED APPLICATIONS

This Application is a continuation-in-part of U.S. patent application Ser. No. 19/080,493, filed Mar. 14, 2025, and entitled “METHODS FOR ADMINISTERING A COMPANION SUPPLEMENT FOR DOGS AND RELATED COMPOSITIONS”, the entirety of which is herein incorporated by reference.

### FIELD

The present disclosure relates to methods for developing companion supplements for dogs, related compositions, and systems.

### BACKGROUND

Proper canine nutrition provides health benefits that target best-available health outcomes, such as skin and coat integrity, cognitive development, visual development, joint support, digestive support, immune support, stress reduction, microbiome balance, mental wellbeing, weight management, and combinations thereof.

### SUMMARY

Some embodiments relate to a method of developing a companion supplement tailored to a dog's stage of life and a dog's nutritional requirements, the method comprising choosing a base food format, wherein the base food format comprises base food ingredients; determining post-cook characteristics of the base food format, wherein the post-cook characteristics are selected from the group consisting of nutrient levels, temporal nutrient degradation modeling across an expected shelf-life of the base food format, bioavailability, digestibility, nutrient antagonisms, chemical form, oxidation losses over a shelf-life of the base food format, stability over a shelf-life of the base food format, breed-specific risk factors, and combinations thereof; preparing a companion supplement based, at least in part, on the post-cook characteristics of the base food format, wherein the companion supplement comprises companion supplement ingredients, and wherein the companion supplement ingredients are selected from one of a limiting amino acid that is not present in the base food format, one or more enzymes calibrated to a protein:fat:carbohydrate ratio of the base food format, and combinations thereof; determining post-production characteristics of the companion supplement; and combining nutrient values of the base food format and the companion supplement to ensure safety and efficacy, and wherein the companion supplement and the base food format, when administered together, are sufficiently designed to provide the dog with a nutritionally optimal feeding system for the dog based on the dog's stage of life and nutritional requirements. In some embodiments, the base food format is selected from non-pressure agglomeration dry pellets, extruded dry pellets, freeze-dried food, air-dried food, cold-pressed food, and raw frozen food.

Some embodiments relate to a method for developing and producing a companion supplement tailored to a dog's stage of life and a dog's nutritional requirements for co-administration with a base food format, the method comprising: selecting a base food formats comprising base food ingredients; manufacturing a representative commercial production batch under a standard production process for that format; determining a post-cook characterization set for the representative commercial production batch; identifying nutrients for a companion supplement for the base food format, the nutrients based on at least one of: (a) sensitivity to thermal, pressure, and mechanical stresses during base food processing; (b) regulatory restrictions on inclusion in pet food; (c) functional benefits supporting optimal health beyond baseline nutritional adequacy; (d) a nutritional composition of the base food; and (e) any combinations thereof; developing the companion supplement for co-administration with the base food format, the companion supplement comprising companion supplement ingredients selected to provide the dog with essential nutrients to ensure adequate intake of essential nutrients when co-administered, and wherein the companion supplement ingredients are selected from one of a limiting amino acid that is not present in the base food format, one or more enzymes calibrated to a protein:fat:carbohydrate ratio of the base food format, and combinations thereof; producing the companion supplement according to the developed companion supplement and packaging the companion supplement physically separate from the base food; and providing administration instructions and labeling that identify compatibility of the packaged companion supplement with the base food format and specify co-administration at feeding, wherein the companion supplement is developed from the determined post-cook characterization set of the representative commercial production batch without reformulating the base food format, and when co-administered with the base food format, the companion supplement and the base food format are sufficiently designed to provide the dog with a nutritionally optimal feeding system based on the dog's stage of life and nutritional requirements.

Some embodiments relate to a method for developing and producing a companion supplement tailored to a dog's stage of life and a dog's nutritional requirements for co-administration with manufactured base food formats, the method comprising: selecting a set of base food formats, each comprising base food ingredients; manufacturing, for each selected base food format, a representative commercial production batch under a standard production process for that format; determining a post-cook characterization set for each representative commercial production batch; identifying nutrients for a companion supplement for each set of base food formats, wherein the identified nutrients are based on at least one of: (a) sensitivity to thermal, pressure, and mechanical stresses during base food processing; (b) regulatory restrictions on inclusion in pet food; (c) functional benefits supporting optimal health beyond baseline nutritional adequacy; (d) a nutritional composition of the base food; and (e) any combinations thereof; developing a companion supplement for co-administration with a corresponding base food format, the companion supplement comprising companion supplement ingredients selected to provide the dog with essential nutrients to ensure adequate intake of essential nutrients when co-administered, and wherein the companion supplement ingredients are selected from one of a limiting amino acid that is not present in the base food format, one or more enzymes calibrated to a protein:fat:carbohydrate ratio of the base food format, and combinations thereof; producing the companion supplement according to the developed companion supplement and packaging the companion supplements physically separate from their respective base food sets; and providing administration instructions and labeling that identify compatibility of the packaged companion supplement with the corresponding base food format and specify co-administration at feeding; wherein the companion supplement is developed from the determined post-cook characterization set of the representative commercial production batch without reformulating the base food format, and when co-administered with the base food format, the companion supplement and the base food format are sufficiently designed to provide the dog with a nutritionally optimal feeding system based on the dog's stage of life and nutritional requirements.

In some embodiments, the base food format is selected from non-pressure agglomeration dry pellets, extruded dry pellets, freeze-dried food, air-dried food, cold-pressed food, and raw frozen food.

In some embodiments, a companion supplement of the present disclosure is not exposed to heat during manufacturing. In some embodiments, the companion supplement of the present disclosure is a soft-chew. In some embodiments, the companion supplement of the present disclosure is freeze-dried. In some embodiments, a companion supplement of the present disclosure is sufficiently designed to complement a base food format of the present disclosure and support multiple systems at once, including those selected from at least one of: cognitive and mood support (ingredients selected to promote attentiveness, engagement, and calmer daily behavior); mobility and flexibility support (formulated to help dogs move more freely and comfortably); skin and coat nourishment (targeted nutrients that can enhance softness, shine, and overall appearance); gut health and digestion (a combination of functional ingredients intended to promote stool quality, regularity, and digestive comfort); and immune and vitality support (a system-wide approach aimed at helping dogs feel brighter, more resilient, and ready for each day); and combinations thereof.

In some embodiments, bioavailability (the proportion of nutrients that enter the bloodstream and can be used by the body's tissues) is determined by testing a dog's feces after being on a nutritional plan of the present disclosure which includes co-administration of a base food format and a tailored companion supplement. In some embodiments, digestibility (a measurement of how much of a food's nutritional value can be absorbed into the bloodstream) is determined by testing a dog's feces after being on a nutritional plan of the present disclosure which includes co-administration of a base food format and a tailored companion supplement. For digestibility calculations, the total daily amount of food consumed minus the amount that's in the feces is calculated. A higher digestibility percentage indicates that more of the food's nutritional value is being absorbed by the body. Foods with high bioavailability and digestibility are more likely to support a pet's health and wellbeing.

In some embodiments, a post-cook characterization set of a base food format of the present disclosure includes determining the dry-matter nutrient levels of the base food and can include determining levels of at least one or more of the following: moisture, dry matter, crude protein, crude fat, fiber, starch (gelatinized), total starch, minerals, amino acids, crude fiber, and ash.

Some embodiments relate to a system comprising: a packaged base food comprising base food ingredients, wherein the base food ingredients are selected from one of protein from a protein source, a chicken fat, vitamins, and minerals, and combinations thereof, wherein the base food format is selected from non-pressure agglomeration pellets, extruded pellets, freeze-dried food, air-dried food, cold-pressed food, or raw frozen food; and a separately packaged tailored companion supplement comprising ingredients selected from one of an Omega-3 fatty acid, a probiotic, a limiting amino acid not present in the base food format, an antioxidant, and heat-sensitive vitamins not present in the base food format, and combinations thereof, the supplement having been developed from post-cook characterization of the base food.

Some embodiments relate to a kit comprising: (a) a packaged base food comprising base food ingredients, wherein the base food ingredients include protein from a protein source, a chicken fat, vitamins, and minerals, wherein the base food format is selected from non-pressure agglomeration pellets, extruded pellets, freeze-dried food, air-dried food, cold-pressed food, or raw frozen food; and (b) a separately packaged companion supplement comprising an Omega-3 fatty acid, a probiotic, an antioxidant, and heat-sensitive vitamins that are not present in the base food, the supplement having been developed from characterization of the base food; and (c) labeling specifying co-administration at feeding.

Some embodiments relate to a method for a well-rounded nutritional plan for a dog. The method comprises administering a quantity of a grain-inclusive base food format to a dog, wherein the quantity is determined based on the dog's body weight and life stage and administering a quantity of a grain companion supplement to the dog, wherein the quantity is determined based on the dog's body weight. In some embodiments, a grain-inclusive base food format of the present disclosure is referred to as “KibbleX composition”. In some embodiments, a grain companion supplement of the present disclosure is referred to as “a KibbleX companion supplement”.

Some embodiments relate to a method for a well-rounded nutritional plan for a dog. The method comprises administering between about 2 ounces to 6 ounces of a KibbleX composition to a dog. The KibbleX composition comprises about 20% to 35% protein from a protein source on a total weight basis of the KibbleX composition. The protein source comprises at least one of a chicken meal, a turkey meal, lamb meal, or any combination thereof. The KibbleX composition comprises about 30 to 55% grains on a total weight basis of the KibbleX composition. The KibbleX composition comprises about 5 to 20% chicken fat on a total weight basis of the KibbleX composition. The method comprises administering between about 3 to 5 grams of a grain companion supplement. The grain companion supplement comprises vitamins comprising at least one of Vitamin C, a Vitamin E, or any combination thereof. The grain companion supplement comprises a skin and coat blend comprising at least one of a zinc, a DHA, a biotin, or any combination thereof. The grain companion supplement comprises amino acids comprising at least one of a L-Carnitine, a tryptophan, or any combination thereof. The grain companion supplement comprises a joint support blend comprising a glucosamine HCl, a chondroitin sulfate, a methylsulfonylmethane (MSM), collagen peptides, a manganese, or any combination thereof. The grain companion supplement comprises a digestive support blend comprising at least one of an inulin, a fructooligosaccharides a mannanoligosaccharides Beta-glucans, arabinogalactans, a ginger root extract, a bromelain, a lipase, a protease, an amylase, or any combination thereof. The companion supplement comprises a probiotic blend. The grain companion supplement comprises a herbal blend comprising at least one of a siliphos, a curcumin, an ashwagandha KSM-66, a kelp powder, or any combination thereof. The grain companion supplement comprises at least one of a choline, a Coenzyme Q10, or any combination thereof.

Some embodiments relate to a method for a well-rounded nutritional plan for a dog. The method comprises administering a quantity of a grain-free base food format to a dog, wherein the quantity is determined based on the dog's body weight and life stage and administering a quantity of a grain-free companion supplement to the dog, wherein the quantity is determined based on the dog's body weight. In some embodiments, a grain-free base food format of the present disclosure is referred to as “grain-free KibbleX composition”. In some embodiments, a grain-free companion supplement of the present disclosure is referred to as “a KibbleX grain-free companion supplement”.

Some embodiments relate to a method for a well-rounded nutritional plan for a dog. The method comprises administering between about 2 ounces to 6 ounces of a grain-free KibbleX composition to a dog. The grain-free KibbleX composition comprises about 35% to 55% protein from a protein source on a total weight basis of the KibbleX composition. The protein source comprises at least one of a chicken meal, a turkey meal, peas, or any combination thereof. The KibbleX composition comprises about 15% to 40% of a carbohydrate source on a total weight basis of the kibble composition. The carbohydrate source comprising at least one of white potatoes, sweet potatoes, chickpeas, or any combination thereof. The grain-free KibbleX composition comprises about 5% to 20% chicken fat on a total weight basis of the KibbleX composition. The method comprises administering between about 3 to 5 grams of a grain-free companion supplement. The grain-free companion supplement comprises vitamins comprising at least one of Vitamin C, a Vitamin E, or any combination thereof. The grain-free companion supplement comprises a skin and coat blend comprising at least one of a zinc, Omega-3 fatty acids, a biotin, or any combination thereof. The grain-free companion supplement comprises amino acids comprising at least one of a L-Carnitine, a tryptophan, or any combination thereof. The grain-free companion supplement comprises a joint support blend comprising a glucosamine HCl, a chondroitin sulfate, a methylsulfonylmethane (MSM), collagen peptides, a manganese, or any combination thereof. The grain-free companion supplement comprises a digestive support blend comprising at least one of an inulin, a fructooligosaccharides a mannanoligosaccharides Beta-glucans, arabinogalactans, a ginger root extract, a bromelain, a lipase, a protease, an amylase, or any combination thereof. The grain-free companion supplement comprises a probiotic blend. The grain-free companion supplement comprises a herbal blend comprising at least one of a siliphos, a curcumin, an ashwagandha KSM-66, a kelp powder, or any combination thereof. The grain-free companion supplement comprises at least one of a choline, a Coenzyme Q10, or any combination thereof. The grain-free companion supplement is tailored to complement the grain-free kibble composition.

In some embodiments, determining post-cook characteristics of a base food format comprises measuring dry-matter nutrient levels of a representative commercial production batch within about one week to about four weeks after manufacturing and moisture removal, and further includes generating temporal degradation curves for vitamins, fatty acids, and amino acids across an expected shelf-life using first-order kinetics.

In some embodiments, preparing the companion supplement comprises formulating to correct the quantified per-nutrient shortfalls such that modeled co-administration meets stage-of-life minimums at a modeled expiration time and remains below applicable safety maximums at both the time of manufacture and at end of the expected shelf-life.

In some embodiments, manufacturing of the companion supplement is performed without exposing the companion supplement ingredients to temperatures or pressures that substantially reduce potency of heat-labile ingredients, including manufacturing without exposure to temperatures above 50° C.

In some embodiments, the companion supplement is a soft chew or a freeze-dried composition produced under low-temperature conditions sufficient to preserve the activity of probiotics, natural enzymes, omega-3 fatty acids, and heat-sensitive vitamins.

In some embodiments, the companion supplement is packaged physically separate from the base food format and includes labeling that identifies the compatible base food format(s) and specifies co-administration at feeding.

In some embodiments, the method further comprises verifying, by laboratory analysis and model-based calculations, that when the base food format and the companion supplement are co-administered according to the administration instructions, combined nutrient intake meets stage-of-life minimums within predetermined tolerances at both the time of manufacture and at modeled end of shelf-life, and does not exceed safety maximums under worst-case combined-fresh assumptions.

In some embodiments, antioxidant species and levels in the companion supplement are selected based on modeled oxidation losses of the base food format so that the nutritionally optimal feeding system achieves a target shelf-life for co-administration.

In some embodiments, determining post-cook characteristics includes identifying a limiting essential amino acid in the finished base food after processing, and formulating the companion supplement to supply the identified limiting amino acid in an amount effective to improve overall essential amino acid bioavailability when co-administered.

In some embodiments, enzyme selection and dosing in the companion supplement are calibrated to the actual post-cook protein:fat:carbohydrate ratio of the base food format to improve macronutrient digestion and absorption.

In some embodiments, a method of the present disclosure further comprises creating a base-format supplement mapping that associates each characterized base food format with a corresponding companion supplement formulation, and providing labeling that identifies the mapping to end users.

In some embodiments, a method of the present disclosure ensures that combined nutrient levels of the base food format and the companion supplement never exceed applicable safety maximums when both products are fresh while meeting stage-of-life minimums at modeled expiration.

In some embodiments, the administration instructions specify that the companion supplement is co-administered within about one second to about five minutes of feeding the base food format to synchronize digestive availability of targeted nutrients.

In some embodiments, determining post-cook characteristics of the base food format includes quantifying oxidation losses of polyunsaturated fatty acids, stability profiles of fat-soluble vitamins, and matrix-dependent bioavailability effects due to processing-induced Maillard reactions.

In some embodiments, the verifying step includes stability testing of the companion supplement under labeled storage conditions and confirmation that labeled potencies, when combined with the modeled end-of-shelf-life potencies of the base food format, satisfy life-stage requirements.

In some embodiments, the post-cook characterization of the base food format is performed immediately after microbial release and moisture normalization of the representative commercial production batch to capture peak potency for degradation modeling.

The methods of the various embodiments can be used to develop companion supplements for warm-blooded higher-level vertebrate animals, such as canines and felines. While the various embodiments described methods for developing and preparing companion supplements for canines to be used with a canine base food format, it should be appreciated that similar methods can be used for developing and preparing companion supplements for felines to be used with a feline base food.

### DRAWINGS

FIG. 1 is a flowchart of a method, according to some embodiments.

FIG. 2 is a flowchart of a method, according to some embodiments.

FIG. 3 is a flowchart of a method, according to some embodiments.

FIG. 4 is a flowchart of a method, according to some embodiments.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Any prior patents and publications referenced herein are incorporated by reference in their entireties.

The Association of American Feed Control Officials (AAFCO) is an independent organization that has been guiding state, federal and international feed regulators with ingredient definitions, label standards and laboratory standards, while supporting the health and safety of people and animals. There are plenty of factors to consider when choosing the right dog food or cat food, but one thing that all veterinarians agree on is that whichever pet food you select, it needs to be AAFCO-compliant. AAFCO-recommended dog foods and cat foods have an AAFCO statement printed on the packaging. The AAFCO statement explains whether the food contains essential nutrients, how that was determined, and which life stage the food is appropriate for. It basically lets you know that the food is complete and balanced for a particular life stage, meaning the food is nutritionally adequate and has necessary nutrients in the correct amounts and ratios.

Nutritional adequacy standards established by the AAFCO must be met or exceeded for a pet food to be marketed as “complete and balanced” for a certain life stage. The AAFCO does not directly test, regulate, approve, or certify pet foods to make sure that they meet the standard requirements. Instead, they establish guidelines for ingredient definitions, product labels, feeding trials, and laboratory analyses of the nutrients that go into pet food. AAFCO sets nutritional standards for dog food, requiring companies to report nutrient levels on the label's “as fed” (AF) basis, but their profiles (minimums for protein, etc.) are set on a dry matter (DM) basis, meaning all moisture is removed from comparison. To compare wet food (high moisture) to dry food (low moisture), you must calculate the “as fed” nutrients to DM basis by dividing the AF nutrient percentage by (100% minus the AF moisture percentage), revealing the true concentration, which is important for evaluating food quality and meeting AAFCO standards.

Pet food companies then use third-party testing agencies to analyze their foods according to AAFCO guidelines. AAFCO guidelines for pet food labels include: product and brand name; species of animal that the food is intended for; net quantity; guaranteed analysis; ingredient list; nutritional adequacy statement (complete and balanced statement); feeding directions; and name and location of the manufacturer. Ingredients must be listed in order of predominance by weight, on an “as formulated” basis. Pet food ingredient lists are required to be based on pre-cook weight including moisture content, not the dry matter composition of the finished product. This regulatory requirement, while standardized, can create unexpected nutritional variations and results in pet food that is nutritionally adequate and not nutritionally optimal.

For example, when “fresh chicken” appears as the first ingredient on a pet food with an AAFCO ingredient list, fresh chicken typically contains 70-75% moisture and, after extrusion, can contribute less dry matter than the ingredients listed third or fourth. For example, when “fresh chicken” is listed as the 1^{st} ingredient it can be measured as 1000 g pre-cook and result in 250 g dry matter post-processing. When “chicken meal” is listed as the 3^{rd} ingredient it can be measured as 500 g pre-cook and result in 450 g dry matter post-processing. When “corn meal” is listed as the 2^{nd} ingredient it can be measured as 600 g pre-cook and result in 540 g dry matter post-processing. In this example, the actual dry matter ranking would be corn (540 g), chicken meal (450 g), fresh chicken (250 g). This example illustrates why post-cook dry-matter post-processing analysis results in a more accurate nutritional formulation—the actual composition can differ significantly from what the ingredient list suggests.

The Food and Drug Administration (FDA) makes sure the ingredients used in pet food are safe and have a purpose. Some ingredients, such as meat, poultry, and grains, are deemed safe. Other substances, including vitamins, minerals, flavorings, and preservatives, may be generally recognized as safe for an intended use.

States may have their own regulations as well. Many states follow models based on AAFCO recommendations. State feed laws require ingredients to either be a “common or usual name,” approved by the state official, or defined and listed in AAFCO's Official Publication. Only AAFCO-approved, Generally Recognized as Safe (GRAS), or FDA-permitted ingredients may be included in pet food. This regulatory framework, while ensuring safety and consistency, creates an unintended barrier to nutritional optimization.

Because vitamins degrade during base food processing and storage, feed/pet-food formulators routinely add overages to ensure label guarantees are met at the end of shelf life; margins of safety can be 20-100% (or higher for certain B-vitamins), depending on the process and storage conditions. Typically, to ensure compliance with AAFCO minimums at expiration, manufacturers have been known to add 20-50% excess of degradable vitamins at the time of manufacture. AAFCO emphasizes batch-to-batch variation and the need for laboratory analysis of the finished product to establish reliable guarantees. Multi-state enforcement data show measured nutrients often differ from guaranteed values, confirming that the guarantee is a legal minimum/maximum, not a time-point-specific “as-fed” constant.

When different entities make food and supplements without access to each other's complete post-cook dry matter post-processing data and shelf-life degradation curves, consumers- and independent supplement makers-cannot know the combined, as-fed levels on any given day. The only way to deliver safe, optimal nutrition across shelf life is unified formulation by a single entity that (a) performs post-cook nutritional analysis of the dry matter food, (b) models degradation over a shelf-life of the food, and (c) co-formulates a companion supplement to that specific food so as to develop a nutritionally optimal feeding system based on the dog's stage of life and nutritional requirements.

Multivitamin or general-purpose supplements are sometimes added to a pet's nutrition plan. The nutrition plan including a base food composition followed up with a multivitamin or general-purpose supplement can create nutritional problems, such as harmful excesses of vitamins and minerals and nutritional deficiencies in the pet diet.

The methods provided herein overcome these challenges by providing a custom-designed companion supplement tailored to complement a base food format. These custom-designed companion supplements result in a nutrition plan that is nutritionally optimal and targets best-available health outcomes, not just nutritionally adequate as established by the AAFCO guidelines. Nutritionally optimal considerations include, but are not limited to, bioavailability, digestibility, and nutrient antagonisms, chemical form of ingredients, processing-induced heat/oxidation losses, stability over the entire shelf life of a pet food, and individual or breed-specific risk factors. These optimization factors often require delivery routes or ingredients not feasible within a conventional pet food matrix governed by AAFCO feed rules.

In some embodiments, a base food format of the present disclosure is extruded food, referred to as “Kibble”. Extruded kibble is made through a high-heat cooking process that enhances digestibility and shelf life, but has also been shown to lead to 20-50% nutrient loss. In some embodiments, a base food format of the present disclosure is a freeze-dried food. In some embodiments, a base food format of the present disclosure is an air-dried food. In some embodiments, a base food format of the present disclosure is a slow-roasted food. In some embodiments, a base food format of the present disclosure is a raw frozen food. In some embodiments, a base food format of the present disclosure is a gently cooked food.

Each base food format selected creates fundamentally different nutritional profiles requiring format-specific companion supplements disclosed herein. The result is the development of base foods and companion supplements that can be co-administered and provide a nutritionally optimal pet nutritional plan. This tailored nutritional plan can result in heat-labile and pressure-sensitive nutrients delivered intact at feeding time because they are present in a companion supplement that has not been exposed to heat or pressure manufacturing processes as compared with a base food that might be exposed to heat or pressure manufacturing processes. Such heat-labile and pressure-sensitive nutrients that can be included in a companion supplement of the present disclosure but are not included in a base food format of the present disclosure are selected from one of probiotics (which might be destroyed above 50° C.), omega-3 fatty acids (which are subject to oxidation during extrusion), natural enzymes (which can be denatured above 48° C.), and heat-sensitive B Vitamins, and combinations thereof.

In some embodiments, characteristics of a base food of the present disclosure are determined, which include, but are not limited to, a post-cook analysis of the nutrient levels of the base food. Additional characteristics of the post-cook base food can include temporal nutrient degradation modeling across an expected shelf-life of the base food ingredients, oxidation losses of the base food ingredients over a shelf-life, stability of the base food ingredients over a shelf-life, and breed-specific risk factors, and combinations thereof. Based on the characterization, ingredients to be formulated to develop a specific companion supplement for a specific base food can be developed. In some embodiments, measuring actual nutritional content after processing (post-cook) includes measuring moisture, protein, fat, fiber, ash, amino acid profile, and fatty acid levels. In some embodiments, based on the characterization of the base food, ingredients that can be formulated in the companion supplement that are not present in the base food but would provide a dog with a nutritionally optimal feeding plan, include, enzymes that can help break down the protein and/or fat present in the base food; additional amino acids; heat-labile and pressure-sensitive vitamins; probiotics; polyphenols; antioxidants; glucosamine. Some of the ingredients present in a companion supplement of the present disclosure are either not present in a base food because it has been manufactured using high pressure and shear that would destroy it, or are present in a base food but, once the base food undergoes processing using a high pressure or high shear process (e.g., extrusion) would be partially or completely destroyed. In an embodiment, the post-cook analysis of the base food identifies limiting amino acids and therefore a specific companion supplement can be developed that provides targeted supplementation to enhance bioavailability of all essential amino acids.

Protein bioavailability is not determined by total protein content. It is limited by the essential amino acid present in the smallest amount relative to the body's needs. This “limiting amino acid” constrains the body's ability to utilize all other amino acids for protein synthesis. Even if a dog's diet is rich in nine of the ten essential amino acids, a shortage of just one means the body cannot effectively build the proteins it requires. The excess amino acids are wasted. The limiting amino acid cannot be determined from the formula. It can only be identified by analyzing the finished food after processing. This is because different amino acids degrade at different rates during extrusion, heat, pressure, and Maillard reactions affect amino acid availability, pre-cook formulas cannot predict post-cook amino acid profiles, and only laboratory analysis of the finished product reveals the actual limiting amino acid.

A companion supplement of the present disclosure can include targeted amounts of ingredients selected from one of vitamins (for example Vitamin C, Vitamin E, and combinations thereof); a skin and coat blend (for example zinc, Omega-3 fatty acids, a biotin, and combinations thereof; amino acids (for example a L-Carnitine, a tryptophan, and combinations thereof; a joint support blend (for example a glucosamine HCl, a chondroitin sulfate, a methylsulfonylmethane (MSM), collagen peptides, a manganese, and combinations thereof; a digestive support blend (for example an inulin, a fructooligosaccharides a mannanoligosaccharides Beta-glucans, arabinogalactans, a ginger root extract, a bromelain, a lipase, a protease, an amylase, and combinations thereof); a probiotic blend; an herbal blend (for example a siliphos, a curcumin, an ashwagandha KSM-66, a kelp powder, and combinations thereof; a choline; an antioxidant (for example Coenzyme Q10); and combinations thereof.

In some embodiments, a feeding trial can be performed to demonstrate measurable efficacy of the co-administration of a base food of the present disclosure with a tailored companion supplement of the present disclosure across eight biological support systems. The trial may generate evidence at three levels: functional outcomes (crude protein digestibility, fecal quality, coat condition), biomarker changes (blood chemistry, inflammation, metabolic markers), and mechanistic evidence (microbiome analysis, short-chain fatty acid production). The trial may collect the following samples: fecal (for microbiome analysis, digestibility, Lactobacillus abundance, butyrate, short-chain fatty acids), blood (CBC, metabolic panel, fatty acid profile, high-sensitivity CRP, cortisol, Vitamin E, Coenzyme Q10, total antioxidant capacity, zinc, L-cartinine, CTX-II, serum amyloid A, interleukin-6), coat assessments via hair clippings (zinc content, sulfur content, shine, luster, softness, density, dandruff, skin condition), and photos.

The present disclosure provides methods of developing a companion supplement tailored to a dog's stage of life and a dog's nutritional requirements by at least one of: (1) measuring actual nutritional content after processing, not theoretical formulas or misleading precooked weights; (2) accounting for different stability profiles of each base food processing method using first order kinetics; (3) identifying specific deficiencies between processed food and optimal nutrition while respecting AAFCO safety maximums; (4) calculating enzyme ratios based on actual post-cook protein:fat:carbohydrate ratios; and (5) ensuring that combined nutrient levels of the base food and companion supplement never exceed safety maximums even when both products are fresh, while meeting minimums at expiration; and (6) any combinations thereof.

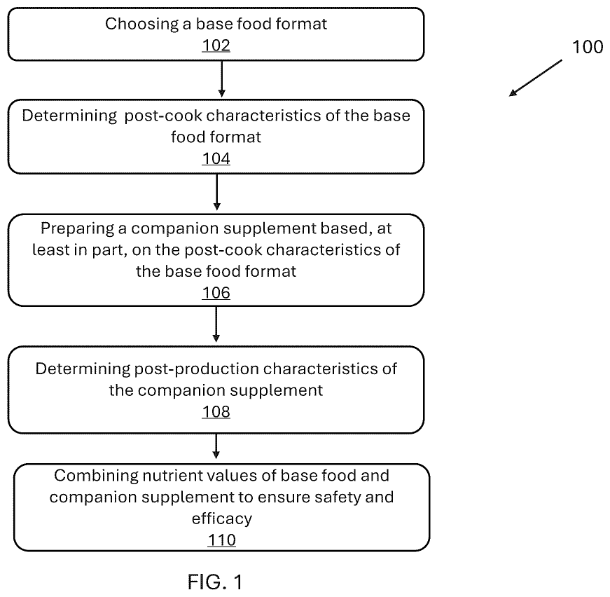

FIG. 1 is a flowchart of a method of developing a companion supplement tailored to a dog's stage of life and nutritional requirements. As shown in FIG. 1, the method 100 comprises the following steps: choosing a base food format (step 102), wherein the base food format comprises base food ingredients; determining post-cook characteristics of the base food format (step 104), wherein the post-cook characteristics are selected from the group consisting of nutrient levels, temporal nutrient degradation modeling across an expected shelf-life of the base food format, bioavailability, digestibility, nutrient antagonisms, chemical form, oxidation losses over a shelf-life of the base food format, stability over a shelf-life of the base food format, and breed-specific risk factors, and combinations thereof; preparing a companion supplement based, at least in part, on the post-cook characteristics of the base food format (step 106), wherein the companion supplement comprises companion supplement ingredients, and wherein the companion supplement ingredients are selected from one of a limiting amino acid that is not present in the base food format, one or more enzymes calibrated to a protein:fat:carbohydrate ratio of the base food format, and combinations thereof; determining post-production characteristics of the companion supplement (step 108); and combining nutrient values of the base food format and the companion supplement to ensure safety and efficacy (step 110), and wherein, the companion supplement and the base food format, when administered together, are sufficiently designed to provide the dog with a nutritionally optimal feeding system for the dog based on the dog's stage of life and nutritional requirements.

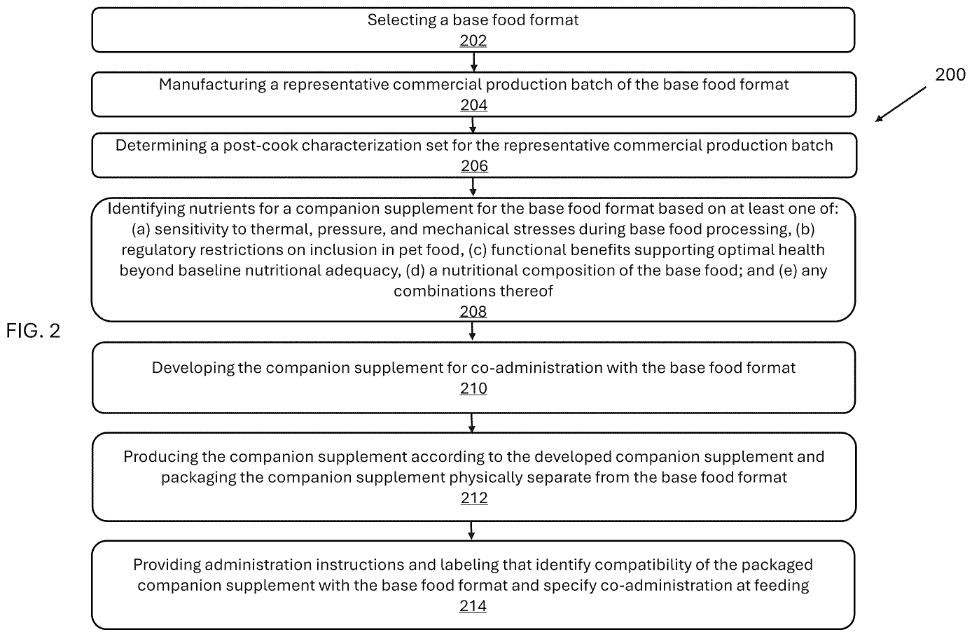

FIG. 2 is a flowchart of a method for developing and producing a companion supplement tailored to a dog's stage of life and nutritional requirements for co-administration with a base food format. As shown in FIG. 2, the method 200 comprises: selecting a base food format comprising base food ingredients (step 202); manufacturing a representative commercial production batch of the base food format (step 204); determining a post-cook characterization set for the representative commercial production batch (step 206); identifying nutrients for a companion supplement for the base food format based on at least one of: (a) sensitivity to thermal, pressure, and mechanical stresses during base food processing; (b) regulatory restrictions on inclusion in pet food; (c) functional benefits supporting optimal health beyond baseline nutritional adequacy; (d) a nutritional composition of the base food; and (e) any combinations thereof (208); developing the companion supplement for co-administration with the base food format (step 210), wherein the companion supplement comprises companion supplement ingredients selected to provide the dog with essential nutrients to ensure adequate intake of essential nutrients when co-administered, and wherein the companion supplement ingredients are selected from one of a limiting amino acid that is not present in the base food format, one or more enzymes calibrated to a protein:fat:carbohydrate ratio of the base food format, and combinations thereof; producing the companion supplement according to the developed companion supplement and packaging the companion supplement physically separate from the base food (step 212); and providing administration instructions and labeling that identify compatibility of the packaged companion supplement with the base food format and specify co-administration at feeding (step 214); wherein the companion supplement is developed from the determined post-cook characterization of the representative commercial production batch without reformulating the base food format, and when co-administered with the base food format, the companion supplement and the base food format are sufficiently designed to provide the dog with a nutritionally optimal feeding system based on the dog's stage of life and nutritional requirements.

In some embodiments, the base food ingredients include protein from a protein source, a chicken fat, vitamins, and minerals. In some embodiments, the companion supplement ingredients include at least one of an amino acid, an Omega-3 fatty acid, a probiotic, an antioxidant, an enzyme, a heat-sensitive vitamin, and combinations thereof.

In some embodiments, the determining post-cook characteristics of the base food format occurs immediately after manufacturing and moisture removal. Immediately after manufacturing would result in the height of nutritional potency. In some embodiments, after manufacturing the base food format, microbial testing is performed prior to the determining post-cook characteristics of the base food format. In some embodiments, after manufacturing the base food format, microbial testing is performed concurrently with determining post-cook characteristics of the base food format. In some embodiments, the determining post-cook characteristics of the base food format occurs between one day and one week after manufacturing and moisture removal. In some embodiments, the determining post-cook characteristics of the base food format occurs between one week and two weeks after manufacturing and moisture removal. In some embodiments, the determining post-cook characteristics of the base food format occurs between two weeks and three weeks after manufacturing and moisture removal. In some embodiments, the determining post-cook characteristics of the base food format occurs between three weeks and four weeks after manufacturing and moisture removal.

In some embodiments, determining post-cook characteristics of the base food format comprises laboratory measurements performed on finished base food after moisture removal, including dry-matter nutrient analysis of vitamins, minerals, amino acids, crude protein, crude fat, fiber, ash, total and gelatinized starch, and fatty acid profiles using validated methods.

In some embodiments, determining post-cook characteristics of the base food format comprises stability and oxidation assays conducted on the finished base food, including peroxide value, anisidine value, and an oxygen stability index test, and accelerated aging studies to derive first-order degradation rate constants for heat- and oxidation-labile nutrients.

In some embodiments, determining post-cook characteristics of the base food format comprises high-performance liquid chromatography of fat- and water-soluble vitamins, gas chromatography of fatty acids, and amino acid profiling following acid hydrolysis to quantify processing-induced losses.

In some embodiments, determining post-cook characteristics of the base food format comprises measuring water activity and moisture content of the finished base food and correlating these to observed degradation rates for sensitive nutrients across labeled storage conditions.

In some embodiments, determining post-cook characteristics of the base food format comprises in vitro digestibility testing and/or in vivo fecal marker studies to estimate macronutrient digestibility and bioavailability of the finished base food matrix.

In some embodiments, determining post-cook characteristics of the base food format is performed on a representative commercial production batch within about one week to about four weeks after manufacturing and microbial release to capture peak potency for degradation modeling.

In some embodiments, preparing or producing the companion supplement comprises concrete manufacturing steps including blending, cold-granulation, and low-temperature forming to avoid thermal degradation of heat-labile ingredients.

In some embodiments, preparing or producing the companion supplement comprises compression molding of a soft-chew matrix at temperatures not exceeding 50° C., followed by controlled drying to a target water activity of less than 0.65.

In some embodiments, preparing or producing the companion supplement comprises freeze-drying a pre-formed dough at shelf temperatures between about −40° C. and −10° C. and a chamber pressure of less than about 200 mTorr to preserve probiotics, enzymes, and omega-3 fatty acids.

In some embodiments, preparing or producing the companion supplement comprises nitrogen-flushed or vacuum packaging in low-oxygen-transmission-rate films with inclusion of an oxygen absorber or desiccant to maintain labeled potency through shelf life.

In some embodiments, preparing or producing the companion supplement comprises validating probiotic viability by colony-forming unit counts at release and at labeled end of shelf life under the specified storage conditions.

In some embodiments, a nutritionally optimal feeding system of the present disclosure is defined by objective, testable criteria requiring that combined intake from the base food and the companion supplement meets life-stage minimums and does not exceed safety maximums established by recognized authorities at time of manufacture and at modeled end of shelf life.

In some embodiments, the nutritionally optimal criteria are deemed satisfied when combined nutrient intakes for essential vitamins, minerals, amino acids, and fatty acids fall within predetermined tolerances of at least meeting life-stage minimums and remaining below applicable safety maximums, where tolerances are pre-specified as ±10% for minimums and 0% exceedance for maximums.

In some embodiments, compliance with the nutritionally optimal criteria is verified both by analytical testing at product release and by model-based projections using nutrient-specific degradation rate constants to the labeled expiration date.

In some embodiments, worst-case combined-fresh scenarios are evaluated to confirm that co-administration of freshly manufactured base food and companion supplement does not exceed any applicable safety maximum on any nutrient.

In some embodiments, the companion supplement is packaged physically separate from the base food and bears labeling that identifies the compatible base food format or SKU, specifies co-administration at feeding, and provides per-serving dosing instructions.

In some embodiments, the labeling includes a compatibility mapping indicating the base food formats for which the companion supplement has been formulated from post-cook characterization of representative commercial production batches.

In some embodiments, the labeling instructs that the companion supplement be co-administered within about one second to about five minutes of feeding the base food to synchronize digestive availability of targeted nutrients.

In some embodiments, the companion supplement packaging includes storage instructions, a lot code, and a machine-readable link providing lot-specific nutrient potency and co-administration tables for the paired base food.

In some embodiments, a method of the present disclosure further comprises quality control release documentation that records the laboratory measurements, degradation models, tolerances, and verification results supporting the nutritionally optimal criteria for the specific base food and companion supplement pairing.

Amino acids make up the basic building blocks of all proteins. Every dog has the natural ability to manufacture every amino acid they need—except for 10 very special ones. These 10 essential amino acids must come from the diet. Otherwise, a dog could suffer serious health consequences—even death. These 10 essential amino acids are: arginine, histidine, isoleucine, leucine, lysine, methionine, phenylalanine, threonine, tryptophane, and valine. Protein chains are manufactured by a dog's cells in a step-by-step, assembly-line fashion—and if just one amino acid is lacking, the entire process shuts down. This missing nutrient is known as the limiting amino acid. That's because its absence “limits” the process from using any of the other available amino acids altogether. So, every dog food must contain all ten.

In some embodiments, a post-cook analysis of the nutrient levels of a base food of the present disclosure indicates that the base food includes the following nine amino acids: Leucine, Isoleucine, Valine (promotes muscle health), Threonine, Lysine (promotes immune function), and Methionine, Histidine, Arginine, Phenylalanine (for metabolic support). Therefore, the limiting amino acid is tryptophan. Based on this analysis, a companion supplement of the present disclosure would be formulated with tryptophan.

Digestive enzymes are essential for dogs to efficiently digest food and absorb nutrients. They are particularly beneficial for dogs with digestive issues, those who have changes their diet, or those with specific health conditions. In some embodiments, a post-cook analysis of the nutrient levels of a base food of the present disclosure provides for a calculation of enzyme ratios to specific macronutrient profiles, resulting in a determination of enzymes that should be included in a tailored companion supplement of the present disclosure. In some embodiments, these enzymes are selected from at least one of protease, amylase, lipase, bromelain, and combinations thereof.

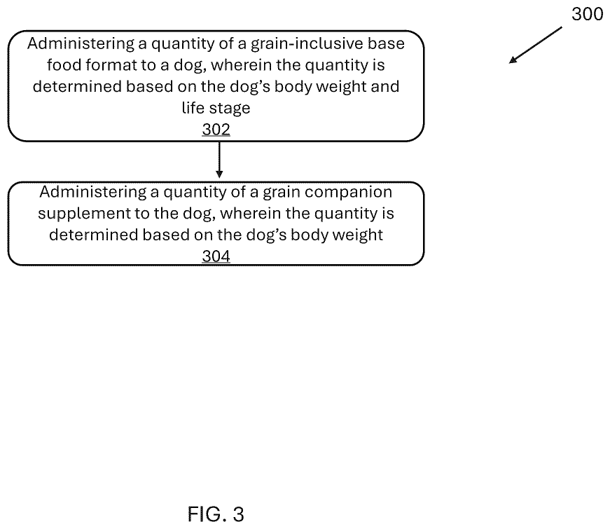

FIG. 3 is a flowchart of an embodiment of a method for a well-rounded nutritional plan for a dog 300, according to some embodiments. As shown in FIG. 3, the method 300 can comprise one or more of the following steps: administering a quantity of a base food format to a dog, wherein the quantity is determined based on the dog's body weight and life stage (302); and administering a quantity of a grain companion supplement to the dog, wherein the quantity is determined based on the dog's body weight (304).

At step 302, in some embodiments, the method comprises administering between about 2 ounces to 6 ounces of a KibbleX composition to a dog, or any range of subrange between about 2 ounces and 6 ounces. For example, in some embodiments, the method comprises administering between about 3 ounces to 5 ounces, 2 ounces to 5 ounces, 2 ounces to 4 ounces, 2 ounces to 3 ounces, 3 ounces to 6 ounces, 4 ounces to 6 ounces, or 5 ounces to 6 ounces of a kibble composition to a dog. At step 302, in some embodiments, the method comprises administering a Kibble X composition to a dog in a quantity based on body weight and life stage, wherein, for example, a 30-pound dog receives between about 1¾ to about 3½ cups as a puppy and between about 1 to 1½ cups as an adult.

In some embodiments, the administering comprises pouring between about 2 ounces to 6 ounces of a KibbleX composition to a dog. In some embodiments, the administering comprises scooping between about 2 ounces to 6 ounces of a KibbleX composition to a dog. In some embodiments, the administering comprises dispensing between about 2 ounces to 6 ounces of a KibbleX composition to a dog. In some embodiments, the administering comprises between about 2 ounces to 6 ounces of a KibbleX composition to a dog. In some embodiments, the administering comprises distributing between about 2 ounces to 6 ounces of a KibbleX composition to a dog. In some embodiments, administering comprises ingesting between about 2 ounces to 6 ounces of a KibbleX composition to a dog.

In some embodiments, the KibbleX composition can comprise a protein from a protein source. In some embodiments, the protein source comprises at least one of a chicken meal, a turkey meal, lamb meal, fish meal, or any combination thereof. In some embodiments, the fish meal can comprise salmon.

In some embodiments, the KibbleX composition can comprise about 20% to 35% protein from a protein source on a total weight basis of the KibbleX composition, or any range or subrange between about 20% and 35%. For example, in some embodiments, the KibbleX composition can comprise about 25% to 30%, 20% to 30%, 20% to 25%, 25% to 35%, or 30% to 35% protein from a protein source on a total weight basis of the KibbleX composition.

In some embodiments, the KibbleX composition comprises grains. In some embodiments, the grains comprises at least a brown rice, an oat groats, pearled barley, or any combination thereof.

In some embodiments, the KibbleX composition can comprise about 30% to 55% grains on a total weight basis of the KibbleX composition, or any range or subrange between 30% and 55%. For example, in some embodiments, the KibbleX composition can comprise about 35% to 50%, 35% to 45%, 30% to 50%, 30% to 45%, 30% to 40%, 30% to 35%, 35% to 55%, 40% to 55%, 45% to 55%, 50% to 55% grains on a total weight basis of the KibbleX composition.

In some embodiments, the KibbleX composition can comprise about 5% to 20% chicken fat on a total weight basis of the KibbleX composition, or any range or subrange between 5% and 20%. For example, in some embodiments, the KibbleX composition can comprise about 10% to 15%, 5% to 15%, 5% to 10%, 10% to 20%, or 15% to 20% chicken fat on a total weight basis of the KibbleX composition.

In some embodiments, the KibbleX composition can be administered at least once per day.

At step 304, in some embodiments, the method comprises administering between about 3 grams to 5 grams of a grain companion supplement. In some embodiments, the administering comprises pouring between about 3 grams to 5 grams of a grain companion supplement. In some embodiments, the administering comprises scooping between about 3 grams to 5 grams of a grain companion supplement. In some embodiments, the administering comprises dispensing between about 3 grams to 5 grams of a grain companion supplement. In some embodiments, the administering comprises between about 3 grams to 5 grams of a grain companion supplement. In some embodiments, the administering comprises distributing between about 3 grams to 5 grams of a grain companion supplement. In some embodiments, administering comprises ingesting between about 3 grams to 5 grams of a grain companion supplement. At step 304, in some embodiments, the method comprises administering a grain companion supplement to the dog in a quantity based on body weight, wherein, for example, a 30-pound dog receives one soft chew.

In some embodiments, a grain companion supplement of the present disclosure can be for all life stages. For example, in some embodiments, the grain companion supplement is provided for puppies developing strong foundations. In some embodiments, the grain companion supplement is provided for adult dogs maintaining optimal health. In some embodiments, the grain companion supplement is provided for senior dogs needing extra support. In some embodiments, the grain companion supplement is provided for active dogs requiring joint support. In some embodiments, the grain companion supplement is provided for dogs experiencing stress or changes. In some embodiments, the grain companion supplement is provided for any dog needing comprehensive nutrition.

In some embodiments, a grain companion supplement of the present disclosure can comprise vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients. The grain companion supplement can have enhanced diet-specific benefits. For example, the grain companion supplement can be formulated to complement a kibble composition. The grain companion supplement can provide a balanced source of nutrients that avoid an excess of nutrients and deliver an appropriate amount of nutrients.

A grain companion supplement of the present disclosure is tailored to complement a KibbleX composition (base food format) of the present disclosure. For example, a grain companion supplement of the present disclosure can be a KibbleX All Life Stage Companion Supplement. The grain companion supplement can be in the form of a soft chew.

A grain companion supplement of the present disclosure can deliver heat-sensitive nutrients at the nutrient's effective potency. Heat-sensitive nutrients can lose potency during the KibbleX base food format manufacturing process. Heat-sensitive ingredients can include Omega-3 fatty acids, collagen peptides, Coenzyme Q10, Vitamin C (Ester-C), and Vitamin E (mixed tocopherols. A grain companion supplement of the present disclosure can have precise dosing of the nutrients to ensure optimal nutrient levels. A grain companion supplement of the present disclosure can include delivered in their most bioavailable forms. A grain companion supplement of the present disclosure can deliver enhanced protein utilization. The protein in a grain companion supplement of the present disclosure can be optimized to the specific KibbleX composition.

In some embodiments, a grain companion supplement of the present disclosure can deliver nutrients not approved by the AAFCO for inclusion in a KibbleX composition. The grain companion supplement can be in compliance with regulatory standards as measured by the AAFCO.

A grain companion supplement of the present disclosure can add nutritional benefits without removing essential nutrients. For example, a grain companion supplement can maintain a calcium-phosphorus ratio.

A grain companion supplement of the present disclosure can comprise vitamins. In some embodiments, the vitamins comprise at least one of Vitamin C, a Vitamin E, or any combination thereof. While the KibbleX composition can provide base antioxidants through vitamin E and selenium, the grain companion supplement can add additional Vitamin E, vitamin C, and astaxanthin from krill oil to create multiple layers of protection against oxidative stress. Vitamin C can protect the fluid inside and between cells, while fat-soluble antioxidants such as vitamin E and astaxanthin can protect cell membranes.

Vitamin C can be a heat-sensitive immune booster and provide vascular health support for collagen production. The grain companion supplement can comprise Vitamin C as Ester-C. Ester-C can be a buffered form for improved gastrointestinal tolerance. In some embodiments, Ester-C provides water-soluble antioxidant protection while also helping to regenerate Vitamin E in the body.

The Vitamin C can be present in an amount of 30 mg to 70 mg per serving, or any range or subrange between 30 milligrams (mg) and 70 mg. For example, in some embodiments, Vitamin C can be present in amount of 35 mg to 65 mg, 40 mg to 60 mg, or 45 mg to 55 mg per serving. In some embodiments, Vitamin C can be present in amount of 35 mg to 70 mg, 40 mg to 70 mg, or 45 mg to 70 mg, 50 mg to 70 mg, 55 mg to 70 mg, 60 mg to 70 mg, or 65 mg to 70 mg per serving. In some embodiments, Vitamin C can be present in amount of 30 mg to 65 mg, 30 mg to 60 mg, 30 mg to 55 mg, 30 mg to 50 mg, 30 mg to 45 mg, 30 mg to 40 mg, or 30 mg to 35 mg per serving.

Vitamin E can be a fat-soluble antioxidant in natural form. Vitamin E can support fatty acid metabolism and protect nutrients from oxidation. Vitamin E can provide anti-inflammatory support and lipid support. Vitamin E can be provided as mixed tocopherols to protect cellular membranes. Provided as mixed tocopherols, Vitamin E can be provided in a natural form with higher bioavailability than synthetic forms.

The Vitamin E can be present in an amount of 10 internation unit (IU) to 50 IU per serving, or any range or subrange between 10 IU and 50 IU. For example, in some embodiments, the Vitamin E can be present in an amount of 15 IU to 45 IU, 20 IU to 40 IU, or 25 IU to 35 IU per serving. In some embodiments, the Vitamin E can be present in an amount of 15 IU to 50 IU, 20 IU to 50 IU, 25 IU to 50 IU, 30 IU to 50 IU, 35 IU to 50 IU, 40 IU to 50 IU, or 45 IU to 50 IU per serving. In some embodiments, the Vitamin E can be present in an amount of 10 IU to 45 IU, 10 IU to 40 IU, 10 IU to 35 IU, 10 IU to 30 IU, 10 IU to 25 IU, 10 IU to 20 IU, or 10 IU to 15 IU per serving.

A grain companion supplement of the present disclosure can comprise a skin and coat blend. The skin and coat blend comprises at least one of a zinc, Omega-3 fatty acids, a biotin, or any combination thereof. The grain companion supplement can elevate skin and coat support without requiring precise dosing of various nutrients. Krill oil can deliver phospholipid-bound omega-3 fatty acids with superior absorption compared to traditional fish oil. Biotin can support keratin production at optimal levels, while zinc carnosine (zinc) can provide specialized support for skin barrier function. The combination of Vitamin E and Vitamin C can provide antioxidant protection and collagen synthesis support. Collagen peptides can provide specific amino acids for skin structure, while MSM, disclosed herein, supports keratin production and helps manage inflammation.

Zinc can be present in the grain companion supplement in the form of zinc carnosine. Zinc carnosine can improve gut health, improve absorption into the body, support stomach lining integrity, and enhance immune support.

Zinc can be present in the grain companion supplement in a precise dose. The zinc can be present in an amount of 20 mg per serving.

Biotin can be present in an amount of 200 micrograms (mcg) to 300 mcg per serving, or any range or subrange between 200 mcg and 300 mcg. In some embodiments, the biotin can be present in an amount of 210 mcg to 290 mcg, 220 mcg to 280 mcg, 230 mcg to 270 mcg, or 240 mcg to 260 mcg per serving. In some embodiments, the biotin can be present in an amount of 210 mcg to 300 mcg, 220 mcg to 300 mcg, 230 mcg to 300 mcg, 240 mcg to 300 mcg, 250 mcg to 300 mcg, 260 mcg to 300 mcg, 270 mcg to 300 mcg, 280 mcg to 300 mcg, or 290 mcg to 300 mcg per serving.

Omega-3 fatty acids can provide anti-inflammatory support and assist in brain development. Omega-3 fatty acids can provide for different nutritional value at various life stages. For example, in some embodiments, for a puppy, Omega-3 fatty acids can assist in brain development. In some embodiments, for an adult dog, Omega-3 fatty acids can enhance skin and coat health. In some embodiments, for a senior dog, Omega-3 fatty acids can improve cognitive function.

Omega-3 fatty acids can be provided in the form of krill oil. Astaxanthin from krill oil can provide for antioxidant properties that complement an existing antioxidant network. Omega-3 fatty acids can be in at least one of a phospholipid form, a triglyceride form, or any combination thereof. Omega-3 fatty acids can be a heat-sensitive nutrient.

The Omega-3 fatty acids can be present in an amount of 200 mg to 300 mg per serving, or any range or subrange between 200 mg and 300 mg. In some embodiments, the Omega-3 Fatty Acids can be present in an amount of 210 mg to 290 mg, 220 mg to 280 mg, 230 mg to 270 mg, or 240 mg to 260 mg per serving. In some embodiments, the Omega-3 Fatty Acids can be present in an amount of 210 mg to 300 mg, 220 mg to 300 mg, 230 mg to 300 mg, 240 mg to 300 mg, 250 mg to 300 mg, 260 mg to 300 mg, 270 mg to 300 mg, 280 mg to 300 mg, or 290 mg to 300 mg per serving.

In some embodiments, the omega-3 fatty acids is present in an amount of 250 mg per serving.

A grain companion supplement of the present disclosure can comprise amino acids. The amino acids comprises at least one of a L-Carnitine, a tryptophan, or any combination thereof. The amino acid can improve protein quality in the grain companion supplement. A dog's diet can utilize about 20 different amin acids, 10 of which can be “essential”. In some embodiments, the grain companion supplement increases the amount of a limiting essential amino acid in a dog's diet to thereby increase the body's ability to utilize amino acids for protein synthesis. By increasing the amount of the limiting essential amino acid in the dog's diet through the grain companion supplement, the availability of all essential amino acids are enhanced as long as the grain companion supplement is given at the same time as the kibble composition, as disclosed herein. In some embodiments, tryptophan is the limiting essential amino acid. The other 9 essential acids comprises histidine, isoleucine, leucine, lysine, methionine, phenylalanine, threonine, valine, and arginine.

In some embodiments, the tryptophan in the grain companion supplement increases the availability of all essential amino acids, thereby improving protein utilization compared to traditional compositions paired with a multivitamin. In some embodiments, the tryptophan in the grain companion supplement presents optimal ratios of the amino acids.

Adding tryptophan to the grain companion supplement helps optimize the absorption environment in the digestive tract, making all amino acids more bioavailable. In some embodiments, tryptophan reduce competitive inhibition between amino acids during absorption, allowing for better utilization of all essential amino acids. Tryptophan can provide neurotransmitter support for production of serotonin.

Tryptophan can be present in an amount of 100 mg to 150 mg per serving, or any range or subrange between 100 mg and 150 mg. For example, in some embodiments, tryptophan can be present in an amount of 105 mg to 145 mg, 110 mg to 140 mg, 115 mg to 135 mg, or 120 mg to 130 mg per serving. In some embodiments, tryptophan can be present in an amount of 105 mg to 150 mg, 110 mg to 150 mg, 115 mg to 150 mg, 120 mg to 150 mg, 125 mg to 150 mg, 130 mg to 150 mg, 135 mg to 150 mg, 140 mg to 150 mg, or 145 mg to 150 mg per serving. In some embodiments, tryptophan can be present in an amount of 100 mg to 145 mg, 100 mg to 140 mg, 100 mg to 135 mg, 100 mg to 130 mg, 100 mg to 125 mg, 100 mg to 120 mg, 100 mg to 115 mg, 100 mg to 110 mg, or 100 mg to 105 mg per serving.

In some embodiments, tryptophan is present in an amount of 125 mg per serving.

A grain companion supplement of the present disclosure can comprise L-Carnitine. L-Carnitine can provide metabolism support. L-Carnitine can be present in the grain companion supplement in a precise dose. The L-Carnitine can be present in an amount of 100 mg per serving.

A grain companion supplement of the present disclosure can comprise a joint support blend. The joint support blend comprises a glucosamine HCl, a chondroitin sulfate, a methylsulfonylmethane (MSM), collagen peptides, a manganese, or any combination thereof. Glucosamine HCl, chondroitin sulfate, MSM, and collagen peptides can provide for stronger joints and mobility in all stages of a dog's life. Joint health can be provided for in the kibble composition natural glucosamine from the chicken meal working with glucosamine HCl and MSM in the grain companion supplement in all stages of a dog's life.

Glucosamine HCl can support cartilage maintenance and enhance joint mobility. Glucosamine HCl can be provided in bioavailable form and can be a building block for cartilage synthesis.

Glucosamine HCl can be present in the grain companion supplement in a precise ratio for optimal absorption compared to sulfate forms. Glucosamine HCl can be present in an amount of 250 mg to 300 mg per serving, or any range or subrange between 250 mg and 300 mg. For example, in some embodiments, glucosamine HCl can be present in an amount of 255 mg to 295 mg, 260 mg to 290 mg, 270 mg to 285 mg, or 275 mg to 280 mg per serving. In some embodiments, glucosamine HCl can be present in an amount of 255 mg to 300 mg, 260 mg to 300 mg, 265 mg to 300 mg, 270 mg to 300 mg, 275 mg to 300 mg, 280 mg to 300 mg, 285 mg to 300 mg, 290 mg to 300 mg, or 295 mg to 300 mg per serving. In some embodiments, glucosamine HCl can be present in an amount of 250 mg to 295 mg, 250 mg to 290 mg, 250 mg to 285 mg, 250 mg to 280 mg, 250 mg to 275 mg, 250 mg to 270 mg, 250 mg to 265 mg, 250 mg to 260 mg, or 250 mg to 255 mg per serving.

MSM can provide for natural anti-inflammatory support and optimize joint and tissue health. MSM can be present in the grain companion supplement in a precise dose. The MSM can be present in an amount of 175 mg per serving.

Chondroitin sulfate can work synergistically with glucosamine HCl to maintain cartilage structure and joint fluid viscosity. Chondroitin sulfate can be present in the grain companion supplement in a precise ratio for optimal absorption. Chondroitin sulfate can be present in an amount of 150 mg to 200 mg per serving, or any range or subrange between 150 mg and 200 mg. For example, in some embodiments, chondroitin sulfate can be present in an amount of 155 mg to 195 mg, 160 mg to 190 mg, 170 mg to 185 mg, or 175 mg to 180 mg per serving. In some embodiments, chondroitin sulfate can be present in an amount of 155 mg to 200 mg, 160 mg to 200 mg, 165 mg to 200 mg, 170 mg to 200 mg, 175 mg to 200 mg, 180 mg to 200 mg, 185 mg to 200 mg, 190 mg to 200 mg, or 195 mg to 200 mg per serving. In some embodiments, chondroitin sulfate can be present in an amount of 150 mg to 195 mg, 150 mg to 190 mg, 150 mg to 185 mg, 150 mg to 180 mg, 150 mg to 175 mg, 150 mg to 170 mg, 150 mg to 165 mg, 150 mg to 160 mg, or 150 mg to 155 mg per serving.

Collagen peptides can be a heat-sensitive nutrient. Collagen peptides can provide for joint structural support. Collagen peptides can improve protein absorption. In some embodiments, collagen peptides provide glycine and proline.

Collagen peptides can be present in an amount of 100 mg to 200 mg per serving, or any range or subrange between 100 mg and 200 mg. For example, in some embodiments, collagen peptides can be present in an amount of 110 mg to 190 mg, 120 mg to 180 mg, 130 mg to 170 mg, or 140 mg to 160 mg per serving. In some embodiments, collagen peptides can be present in an amount of 110 mg to 200 mg, 120 mg to 200 mg, 130 mg to 200 mg, 140 mg to 200 mg, 150 mg to 200 mg, 160 mg to 200 mg, 170 mg to 200 mg, 180 mg to 200 mg, or 190 mg to 200 mg per serving. In some embodiments, collagen peptides can be present in an amount of 100 mg to 190 mg, 100 mg to 180 mg, 100 mg to 170 mg, 100 mg to 160 mg, 100 mg to 150 mg, 100 mg to 140 mg, 100 mg to 130 mg, 100 mg to 120 mg, or 100 mg to 110 mg per serving.

Manganese can be present in an amount of 1 mg to 10 mg per serving, or any range or subrange between 1 mg and 10 mg. For example, in some embodiments, manganese can be present in an amount of 2 mg to 9 mg, 3 mg to 8 mg, 4 mg to 7 mg, or 5 mg to 6 mg per serving. In some embodiments, manganese can be present in an amount of 2 mg to 10 mg, 3 mg to 10 mg, 4 mg to 10 mg, 5 mg to 10 mg, 6 mg to 10 mg, 7 mg to 10 mg, 8 mg to 10 mg, or 9 mg to 10 mg per serving. In some embodiments, manganese can be present in an amount of 1 mg to 9 mg, 1 mg to 8 mg, 1 mg to 7 mg, 1 mg to 6 mg, 1 mg to 5 mg, 1 mg to 4 mg, 1 mg to 3 mg, or 1 mg to 2 mg per serving.

In some embodiments, a grain companion supplement of the present disclosure can comprise a digestive support blend. The digestive support blend can comprise at least one of an inulin, a fructooligosaccharides, a mannanoligosaccharides, Beta-glucans, arabinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend. The prebiotic blend can comprise the inulin, the fructooligosaccharides the mannanoligosaccharides, the Beta-glucans, and the arabinogalactans.

Inulin can provide a long-chain prebiotic fiber supporting sustained fermentation. Inulin can be present in an amount of 50 mg to 150 mg per serving, or any range or subrange between 50 mg and 150 mg. For example, in some embodiments, inulin can be present in an amount of 60 mg to 140 mg, 70 mg to 130 mg, 80 mg to 120 mg, or 90 mg to 110 mg per serving. In some embodiments, inulin can be present in an amount of 50 mg to 140 mg, 50 mg to 130 mg, 50 mg to 120 mg, 50 mg to 110 mg, 50 mg to 100 mg, 50 mg to 90 mg, 50 mg to 80 mg, 50 mg to 70 mg, or 50 mg to 60 mg per serving. In some embodiments, inulin can be present in an amount of 60 mg to 150 mg, 70 mg to 150 mg, 80 mg to 150 mg, 90 mg to 150 mg, 100 mg to 150 mg, 110 mg to 150 g, 120 mg to 150 mg, 130 mg to 150 mg, or 140 mg to 150 mg per serving.

Fructooligosaccharides can provide a short-chain prebiotics for rapid support. Fructooligosaccharides can be present in an amount of 50 mg to 100 mg per serving, or any range or subrange between 50 mg and 100 mg. For example, in some embodiments, fructooligosaccharides can be present in an amount of 55 mg to 95 mg, 60 mg to 90 mg, 65 mg to 85 mg, or 70 mg to 80 mg per serving. In some embodiments, fructooligosaccharides can be present in an amount of 55 mg to 100 mg, 60 mg to 100 mg, 65 mg to 100 mg, 70 mg to 100 mg, 75 mg to 100 mg, 80 mg to 100 mg, 85 mg to 100 mg, 90 mg to 100 mg, or 95 mg to 100 mg per serving. In some embodiments, fructooligosaccharides can be present in an amount of 50 mg to 95 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg per serving.

Mannanoligosaccharides can provide specialized prebiotics to prevent pathogen adhesion. Mannanoligosaccharides can be present in an amount of 25 mg to 75 mg per serving, or any range or subrange between 25 mg and 75 mg. For example, in some embodiments, mannanoligosaccharides can be present in an amount of 25 mg to 75 mg per serving.

Beta-glucans can provide immune-modulating prebiotics. Beta-glucans can be present in an amount of 15 mg to 35 mg per serving, or any range or subrange between 15 mg and 35 mg. For example, in some embodiments, beta-glucans can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 mg per serving.

Arabinogalactans can provide a plant-based prebiotic to support bacteria growth. Arabinogalactans can be present in an amount of 15 mg to 35 mg per serving, or any range or subrange between 15 mg and 35 mg. For example, in some embodiments, arabinogalactans can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 per serving.

Protease can break down proteins into absorbable amino acids. Protease can be present in an amount of 10 mg to 30 mg per serving, or any range or subrange between 10 mg and 30 mg. For example, in some embodiments, protease can be present in an amount of 15 mg to 25 mg, 15 mg to 30 mg, 20 mg to 30 mg, 25 mg to 30 mg, 10 mg to 25 mg, 10 mg to 20 mg, or 10 mg to 15 mg per serving.

Amylase can convert complex carbohydrates into simple sugars. Amylase can be present in an amount of 5 mg to 25 mg per serving, or any range or subrange between 5 mg and 25 mg per serving. For example, in some embodiments, amylase can be present in an amount of 10 mg to 20 mg, 15 mg to 25 mg, 20 mg to 25 mg, 10 mg to 25 mg, 15 mg to 25 mg, or 20 mg to 25 mg per serving.

Lipase can break down fats into fatty acids. Lipase can be present in an amount of 5 mg to 25 mg per serving, or any range or subrange between 5 mg and 25 mg per serving. For example, in some embodiments, lipase can be present in an amount of 10 mg to 20 mg, 15 mg to 25 mg, 20 mg to 25 mg, 10 mg to 25 mg, 15 mg to 25 mg, or 20 mg to 25 mg per serving.

Bromelain is a protein-digesting enzyme. Bromelain can be present in an amount of 10 mg to 30 mg per serving, or any range or subrange between 10 mg and 30 mg. For example, in some embodiments, bromelain can be present in an amount of 15 mg to 25 mg, 15 mg to 30 mg, 20 mg to 30 mg, 25 mg to 30 mg, 10 mg to 25 mg, 10 mg to 20 mg, or 10 mg to 15 mg per serving.

Ginger root extract can provide support for digestive comfort and motility. Ginger root extract can be present in an amount of 15 mg to 35 mg per serving, or any range or subrange between 15 mg and 35 mg. For example, in some embodiments, ginger root extract can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 mg per serving.

In some embodiments, a grain companion supplement of the present disclosure can comprise a probiotic blend. The probiotic blend can be a canine-specific probiotic strains. The probiotic blend can provide colonization support. The probiotic blend can improve digestion function, strengthened immune response, provide optimal gut flora balance, provide diverse prebiotic fibers, and support different bacteria species. For example, in some embodiments, the probiotic blend comprises at least one of lactobacillus, bifidobacterium, Enterococcus faecium, Streptococcus thermophilus, or any combination thereof.

The probiotic blend can be present in an amount of 1 billion colony-forming units (CFU) to 2 billion CFU per serving, or any range or subrange between 1 billion CFU to 2 billion CFU. For example, in some embodiments, the probiotic blend can be present in an amount of 1.1 billion CFU to 1.9 billion CFU, 1.2 billion CFU to 1.8 billion CFU, 1.3 billion CFU to 1.7 billion CFU, or 1.4 billion CFU to 1.6 billion CFU per serving. In some embodiments, the probiotic blend can be present in an amount of 1.1 billion CFU to 2 billion CFU, 1.2 billion CFU to 2 billion CFU, 1.3 billion CFU to 2 billion CFU, 1.4 billion CFU to 2 billion CFU, 1.5 billion CFU to 2 billion CFU, 1.6 billion CFU to 2 billion CFU, 1.7 billion CFU to 2 billion CFU, 1.8 billion CFU to 2 billion CFU, or 1.8 billion CFU to 2 billion CFU per serving. In some embodiments, the probiotic blend can be present in an amount of 1 billion CFU to 1.9 billion CFU, 1 billion CFU to 1.8 billion CFU, 1 billion CFU to 1.7 billion CFU, 1 billion CFU to 1.6 billion CFU, 1 billion CFU to 1.5 billion CFU, 1 billion CFU to 1.4 billion CFU, 1 billion CFU to 1.3 billion CFU, 1 billion CFU to 1.2 billion CFU, or 1 billion CFU to 1.1 billion CFU per serving.

A In some embodiments, a grain companion supplement of the present disclosure cab comprise a herbal blend. The herbal blend can comprise at least one of a siliphos, a curcumin, an ashwagandha KSM-66, a kelp powder, or any combination thereof.

Siliphos, or milk thistle extract can support liver function, enhance detoxification, and provide antioxidant protection. The siliphos can be present in an amount of 25 mg to 75 mg per serving, or any range or subrange between 25 mg to 75 mg. For example, in some embodiments, siliphos can be present in an amount of 30 mg to 70 mg, 35 mg to 65 mg, 40 mg to 60 mg, or 45 mg to 55 mg per serving. In some embodiments, siliphos can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, siliphos can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving.

Curcumin can reduce inflammation, improve immune function, and support joint health. Curcumin can comprise black pepper extract. The black pepper extract can improve bioavailability and ensure the curcumin reaches the liver in active form.

The curcumin can be present in an amount of 25 mg to 75 mg per serving, or any range or subrange between 25 mg to 75 mg. For example, in some embodiments, curcumin can be present in an amount of 30 mg to 70 mg, 35 mg to 65 mg, 40 mg to 60 mg, or 45 mg to 55 mg per serving. In some embodiments, curcumin can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, curcumin can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving.

Ashwagandha KSM-66, or ashwagandha extract can reduce inflammation, support joint health, and improve immune function. Ashwagandha KSM-66 can modulate the effects of stress on liver function while supporting healthy enzyme levels and provide additional antioxidant protection. Ashwagandha KSM-66 can be an adaptogenic herb. Ashwagandha KSM-66 can maintain liver cell integrity during periods of increased stress.

The ashwagandha KSM-66 can be present in an amount of 25 mg to 50 mg per serving, or any range or subrange between 25 mg to 50 mg. For example, in some embodiments, ashwagandha KSM-66 can be present in an amount of 30 mg to 45 mg, 35 mg to 40 mg, 25 mg to 45 mg, 25 mg to 40 mg, 25 mg to 35 mg, 25 mg to 30 mg, 30 mg to 50 mg, 35 mg to 50 mg, 40 mg to 50 mg, or 45 mg to 50 mg per serving.

Kelp powder can provide natural iodine and trace minerals that support overall metabolic function and aid in natural detoxification processes. The kelp powder can be present in an amount of 10 mg to 30 mg per serving, or any range or subrange between 10 mg to 30 mg. For example, in some embodiments, the kelp powder can be present in an amount of 15 mg to 25 mg, 15 mg to 30 mg, 20 mg to 30 mg, 25 mg to 30 mg, 10 mg to 25 mg, 10 mg to 20 mg, 10 mg to 15 mg per serving.

In some embodiments, a grain companion supplement of the present disclosure can comprise at least one of a choline, a Coenzyme Q10, or any combination thereof.

Choline can support vascular health and lipid metabolism. Choline can provide primary neural support for brain function.

The choline can be present in an amount of 25 mg to 75 mg per serving, or any range or subrange between 25 mg to 75 mg. For example, in some embodiments, choline can be present in an amount of 30 mg to 70 mg, 35 mg to 65 mg, 40 mg to 60 mg, or 45 mg to 55 mg per serving. In some embodiments, choline can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, choline can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving,

Coenzyme Q10 can be present in the grain companion supplement in a precise dose. The Coenzyme Q10 can be present in an amount of 15 mg per serving.

In some embodiments, a grain companion supplement of the present disclosure can comprise additional ingredients, such as, Organic Oat Flour, Dried Brewer's Yeast, Glycerin, Fish Oil, Powdered Cooked Chicken, Sunflower Lecithin, Pea Starch, Cane Molasses, Liquid Chicken Flavor, Coconut Oil, Natural Flavor, Mixed Tocopherols.

In some embodiments, a grain companion supplement of the present disclosure can be administered simultaneously (“co-administered”) with administering the KibbleX base food format. In some embodiments, the grain companion supplement can be co-administered about 1 second to 5 minutes of administering the KibbleX base food format, or any range or subrange between about 1 second and 5 minutes. For example, in some embodiments, the grain companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the KibbleX base food format.

Table 1 is an exemplary illustration of ingredients in an embodiment of a KibbleX All Life Stage Companion Supplement of the present disclosure developed in accordance with a method of the present disclosure based on the KibbleX All Life Stage base food having the guaranteed analysis nutritional information listed in Table 2.

TABLE 1
Supplement Facts
Serving Size 1 Soft Chew (4.4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 30 | IU | † |
| tocopherols) | | | |
| Skin & Coat Blend | | | |
| Zinc (as Zinc Carnosine) | 20 | mg | † |
| Omega-3 Fatty Acids | 250 | mg | † |
| (from Krill Oil) | | | |
| Biotin | 250 | mcg | † |
| Amino Acids | 225 | mg | |
| L-Carnitine | 100 | mg | † |
| Tryptophan | 125 | mg | † |
| Joint Support Blend | 780 | mg | |
| Glucosamine HCl | 275 | mg | † |
| Chondroitin Sulfate | 175 | mg | † |
| MSM (Methylsulfonylmethane) | 175 | mg | † |
| Collagen Peptides | 150 | mg | † |
| Manganese | 5 | mg | † |
| Digestive Support Blend | 370 | mg | † |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 25 | mg | † |
| Bromelain | 20 | mg | † |
| Lipase | 15 | mg | † |
| Protease | 20 | mg | † |
| Amylase | 15 | mg | † |
| Probiotic Blend | 2 billion CFU | | † |
| Herbal Blend | 155 | mg | |
| Siliphos | 50 | mg | † |
| Curcumin (with Black | 50 | mg | † |
| Pepper Extract) | | | |
| Ashwagandha KSM-66 | 35 | mg | † |
| Kelp Powder | 20 | mg | † |
| Other Nutrients | | | |
| Choline | 50 | mg | † |
| Coenzyme Q10 | 15 | mg | † |

TABLE 2
Guaranteed Analysis - All Life Stage Dog Base Food

| Crude Protein (min) | 26.0% | |
|---|---|---|
| Crude Fat (min) | 16.0% | |
| Crude Fiber (max) | 5.0% | |
| Moisture (max) | 10.0% | |
| Docosahexaenoic Acid (DHA) (min) | 0.08% | |
| Eicosapentaenoic Acid (EPA) (min) | 0.10% | |
| Calcium (min) | 1.4% | |
| Phosphorus (min) | 1.0% | |
| Vitamin A (min) | 24,000 | IU/kg |
| Vitamin E (min) | 350 | IU/kg |
| Ascorbic Acid (Vitamin C) (min) | 325 | mg/kg |
| Omega-3 Fatty Acids (min) | 0.75% | |
| Omega-6 Fatty Acids (min) | 3.50% | |
| Taurine (min) | 0.12% | |
| Total microorganisms (min) | 10,000,000 | CFU/lb |

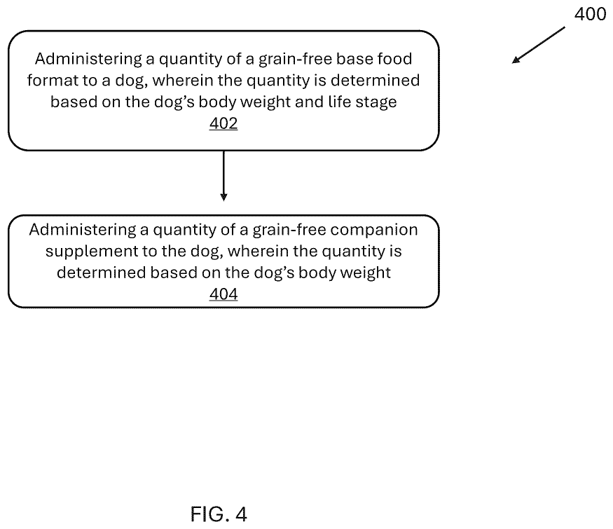

FIG. 4 is a flowchart of an embodiment of a method for a well-rounded nutritional plan for a dog 400, according to some embodiments. As shown in FIG. 4, the method 400 can comprise one or more of the following steps: administering a quantity of a grain-free base food to a dog, wherein the quantity is determined based on the dog's body weight and life stage (step 402); and administering a quantity of a grain-free companion supplement to the dog, wherein the quantity is determined based on the dog's body weight (step 404).

At step 402, in some embodiments, the method comprises administering between about 2 ounces to 6 ounces of a grain-free KibbleX composition to a dog, or any range of subrange between 2 ounces to 6 ounces. At step 402, in some embodiments, the method comprises administering a grain-free Kibble X composition to a dog in a quantity based on body weight and life stage, wherein, for example, a 30-pound dog receives between about 1¾ to about 3½ cups as a puppy and between about 1 to 1½ cups as an adult.

In some embodiments, the administering comprises pouring between about 2 ounces to 6 ounces of a grain-free KibbleX composition to a dog. In some embodiments, the administering comprises scooping between about 2 ounces to 6 ounces of a grain-free KibbleX composition to a dog. In some embodiments, the administering comprises dispensing between about 2 ounces to 6 ounces of a grain-free KibbleX composition to a dog. In some embodiments, the administering comprises between about 2 ounces to 6 ounces of a grain-free KibbleX composition to a dog. In some embodiments, the administering comprises distributing between about 2 ounces to 6 ounces of a grain-free KibbleX composition to a dog. In some embodiments, administering comprises ingesting between about 2 ounces to 6 ounces of a grain-free KibbleX composition to a dog.

The grain-free KibbleX composition can have an improved protein composition compared to traditional grain-free kibble compositions. The grain-free KibbleX composition can comprise an amount of protein more than traditional grain-free kibble composition. The grain-free KibbleX composition can comprise a protein. In some embodiments, the protein is derived from a protein source. In some embodiments, the protein source comprises at least one of a chicken meal, a turkey meal, lamb meal, a fish meal, peas, or any combination thereof as disclosed herein. The peas can provide a plant protein.

In some embodiments, the grain-free KibbleX composition can comprise about 30% to 55% protein from a protein source on a total weight basis of the grain-free KibbleX composition, or any range or subrange between about 30% and 55%. For example, in some embodiments, the grain-free KibbleX composition can comprise about 35% to 50%, 40% to 45%, 35% to 55%, 40% to 55%, 45% to 55%, 50% to 55%, 30% to 50%, 30% to 45%, 30% to 40%, or 30% to 35% protein from a protein source on a total weight basis of the grain-free KibbleX composition.

The grain-free KibbleX composition can provide an alternative carbohydrate source. The grain-free KibbleX composition can comprise a carbohydrate source comprising at least one of white potatoes, sweet potatoes, chickpeas, or any combination thereof. The white potatoes can provide digestible carbohydrates. In some embodiments, the sweet potatoes provide complex carbohydrates. In some embodiments, the chickpeas provide a protein and carbohydrates.

In some embodiments, the grain-free KibbleX composition can comprise about 15% to 40% of a carbohydrate source on a total weight basis of the grain-free KibbleX composition, or any range or subrange between about 15% and 40%. For example, in some embodiments, the grain-free KibbleX composition can comprise about 20% to 35%, 25% to 30%, 15% to 35%, 15% to 30%, 15% to 25%, 15% to 20%, 20% to 40%, 25% to 40%, 30% to 40%, 35% to 40% of a carbohydrate source on a total weight basis of the grain-free KibbleX composition.

In some embodiments, the grain-free KibbleX composition can comprise about 5% to 20% chicken fat on a total weight basis of the KibbleX composition. For example, in some embodiments, the grain-free KibbleX composition can comprise about 10% to 15%, 10% to 20%, 15% to 20%, 5% to 15%, or 5% to 10% chicken fat on a total weight basis of the KibbleX composition.

The grain-free KibbleX composition can provide a fiber matrix. In some embodiments, the grain-free KibbleX composition provides a proprietary fiber and prebiotic mix. In some embodiments, a beet pulp provides fermentable fiber. In some embodiments, the grain-free KibbleX composition provides a plurality of fiber sources to support digestive health.

In some embodiments, the grain-free KibbleX can be administered at least once per day. In some embodiments, the grain-free KibbleX can be administered once per day to 3 times per day, or any range or subrange between once per day and 3 times per day.

At step 404, in some embodiments, the method comprises administering between about 3 to 5 grams of a grain-free companion supplement. In some embodiments, the administering comprises pouring between about 3 to 5 grams of a grain-free companion supplement. In some embodiments, the administering comprises scooping between about 3 to 5 grams of a grain-free companion supplement. In some embodiments, the administering comprises dispensing between about 3 to 5 grams of a grain-free companion supplement. In some embodiments, the administering comprises between about 3 to 5 grams of a grain-free companion supplement. In some embodiments, the administering comprises distributing between about 3 to 5 grams of a grain-free companion supplement. In some embodiments, administering comprises ingesting between about 3 to 5 grams of a grain-free companion supplement. At step 404, in some embodiments, the method comprises administering a grain-free companion supplement to the dog in a quantity based on body weight, wherein, for example, a 30-pound dog receives one soft chew.

The grain-free companion supplement can be for a grain-free for all life stages. For example, in some embodiments, the grain-free companion supplement is provided for puppies developing strong foundations. In some embodiments, the grain-free companion supplement is provided for adult dogs maintaining optimal health. In some embodiments, the grain-free companion supplement is provided for senior dogs needing extra support. In some embodiments, the grain-free companion supplement is provided for active dogs requiring joint support. In some embodiments, the grain-free companion supplement is provided for dogs experiencing stress or changes. In some embodiments, the grain-free companion supplement is provided for any dog needing comprehensive nutrition.

The grain-free companion supplement can be tailored to complement the grain-free kibble composition. For example, in some embodiments, the grain-free companion supplement can be a KibbleX Grain-Fee All Life Stage Companion Supplement. The grain-free companion supplement can be in the form of a soft chew.

The grain-fee companion supplement can optimize protein utilization, provide joint support, provide digestive support, improve skin and coat health, provide metabolic support, and provide immune system support in synergy with the grain-free kibble composition. The grain-free companion supplement can optimize protein utilization. It will be appreciated that any one or more of vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients disclosed herein can comprise the grain-fee companion supplement. It will further be appreciated that the grain-fee companion supplement can comprise the ingredients present in an amount similar, or the same as the companion supplement disclosed herein.

The grain-free companion supplement can deliver heat-sensitive nutrients at the nutrient's effective potency as disclosed herein. The grain-free companion supplement can have precise dosing of the nutrients to ensure optimal nutrient levels.

The grain-free companion supplement can comprise at least one of Vitamin C, a Vitamin E, or any combination thereof. The Vitamin C can be present in an amount of 30 mg to 70 mg per serving, or any range or subrange between 30 milligrams mg and 70 mg as disclosed herein.

The Vitamin E can be present in an amount of 20 IU to 60 IU per serving, or any range or subrange between 20 IU and 60 IU. For example, in some embodiments, the Vitamin E can be present in an amount of 25 IU to 55 IU, 30 IU to 50 IU, or 35 IU to 45 IU per serving. In some embodiments, the Vitamin E can be present in an amount of 25 IU to 60 IU, 30 IU to 60 IU, 35 IU to 60 IU, 40 IU to 60 IU, 45 IU to 60 IU, 50 IU to 60 IU, or 55 IU to 60 IU per serving. In some embodiments, the Vitamin E can be present in an amount of 20 IU to 55 IU, 20 IU to 50 IU, 20 IU to 45 IU, 20 IU to 40 IU, 20 IU to 35 IU, 20 IU to 30 IU, or 20 IU to 25 IU per serving.

The grain-free companion supplement can comprise a skin and coat blend. The skin and coat blend comprises at least one of a zinc, DHA, a biotin, or any combination thereof. The grain-free companion supplement can elevate skin and coat support without requiring precise dosing of various nutrients.

Zinc can be present in the grain-free companion supplement in a precise dose. The zinc can be present in an amount of 15 mg per serving.

Biotin can be present in an amount of 200 micrograms (mcg) to 300 mcg per serving, or any range or subrange between 200 mcg and 300 mcg as disclosed herein.

Omega-3 fatty acids can be present in an amount of 150 mg to 250 mg per serving, or any range or subrange between 150 mg and 250 mg. In some embodiments, the Omega-3 fatty acids can be present in an amount of 160 mg to 240 mg, 170 mg to 230 mg, 180 mg to 220 mg, or 190 mg to 210 mg per serving. In some embodiments, the Omega-3 fatty acids can be present in an amount of 160 mg to 250 mg, 170 mg to 250 mg, 180 mg to 250 mg, 190 mg to 250 mg, 200 mg to 250 mg, 210 mg to 250 mg, 220 mg to 250 mg, 230 mg to 250 mg, or 240 mg to 250 mg per serving. In some embodiments, the Omega-3 fatty acids can be present in an amount of 150 mg to 240 mg, 150 mg to 230 mg, 150 mg to 220 mg, 150 mg to 210, 150 mg to 200 mg, 150 mg to 190 mg, 150 mg to 180 mg, 150 mg to 170 mg, or 150 mg to 160 mg per serving.

In some embodiments, the Omega-3 fatty acids is present in an amount of 200 mg per serving.

The grain-free companion supplement can comprise amino acids. The amino acids comprises at least one of a L-Carnitine, a tryptophan, or any combination thereof.

Tryptophan can be present in an amount of 30 mg to 100 mg per serving, or any range or subrange between 30 mg and 100 mg. For example, in some embodiments, tryptophan can be present in an amount of 40 mg to 90 mg, 50 mg to 80 mg, or 60 mg to 70 mg per serving. In some embodiments, tryptophan can be present in an amount of 40 mg to 100 mg, 50 mg to 100 mg, 60 mg to 100 mg, 70 mg to 100 mg, 80 mg to 100 mg, or 90 mg to 100 mg per serving. In some embodiments, tryptophan can be present in an amount of 30 mg to 90 mg, 30 mg to 80 mg, 30 mg to 70 mg, 30 mg to 60 mg, 30 mg to 50 mg, or 30 mg to 40 mg per serving.

In some embodiments, tryptophan can be the limiting amino acid in the grain-free KibbleX composition. In some embodiments, the grain-free companion supplement provides a precise tryptophan supplementation calculated to optimize protein utilization. In some embodiments, the tryptophan is present in an amount of 70 mg per serving.

The grain-free companion supplement can comprise L-Carnitine. L-Carnitine can provide metabolism support. L-Carnitine can be present in the companion supplement in a precise dose. The L-Carnitine can be present in an amount of 150 mg per serving.

The grain-free companion supplement can comprise a joint support blend. The joint support blend comprises a glucosamine HCl, a chondroitin sulfate, a methylsulfonylmethane (MSM), collagen peptides, a manganese, or any combination thereof. Glucosamine HCl, chondroitin sulfate, MSM, and collagen peptides can provide for stronger joints and mobility in all stages of a dog's life. Joint health can be provided for in the kibble composition natural glucosamine from the chicken meal working with glucosamine HCl and MSM in the companion supplement in all stages of a dog life.

Glucosamine HCl can support cartilage maintenance and enhance joint mobility. Glucosamine HCl can be provided in bioavailable form and can be a building block for cartilage synthesis.

Glucosamine HCl can be present in an amount of 250 mg to 300 mg per serving, or any range or subrange between 250 mg and 300 mg as disclosed herein.

MSM can provide for natural anti-inflammatory support and optimize joint and tissue health. MSM can be present in the companion supplement in a precise dose. The MSM can be present in an amount of 175 mg per serving.

Chondroitin sulfate can be present in an amount of 150 mg to 200 mg per serving, or any range or subrange between 150 mg and 200 mg as disclosed herein.

Collagen peptides can be present in an amount of 100 mg to 200 mg per serving, or any range or subrange between 100 mg and 200 mg as disclosed herein.

Manganese can be present in an amount of 1 mg to 10 mg per serving, or any range or subrange between 1 mg and 10 mg.

The grain-free companion supplement can comprise a digestive support blend. The digestive support blend can comprise at least one of an inulin, a fructooligosaccharides, a mannanoligosaccharides, Beta-glucans, arabinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend.

Inulin can be present in an amount of 50 mg to 150 mg per serving, or any range or subrange between 50 mg and 150 mg as disclosed herein.

Fructooligosaccharides can provide a short-chain prebiotics for rapid support. Fructooligosaccharides can be present in an amount of 50 mg to 100 mg per serving, or any range or subrange between 50 mg and 100 mg as disclosed herein.

Mannanoligosaccharides can be present in an amount of 25 mg to 75 mg per serving, or any range or subrange between 25 mg and 75 mg as disclosed herein.

Beta-glucans can be present in an amount of 15 mg to 35 mg per serving, or any range or subrange between 15 mg and 35 mg as disclosed herein.

Arabinogalactans can be present in an amount of 15 mg to 35 mg per serving, or any range or subrange between 15 mg and 35 mg as disclosed herein.

Bromelain can be present in an amount of 10 mg to 30 mg per serving, or any range or subrange between 10 mg and 30 mg as disclosed herein.

Ginger root extract can be present in an amount of 15 mg to 35 mg per serving, or any range or subrange between 15 mg and 35 mg as disclosed herein.

The grain-free companion supplement can comprise a probiotic blend. The probiotic blend can be a canine-specific probiotic strains. The probiotic blend can provide colonization support. The probiotic blend can improve digestion function, strengthened immune response, provide optimal gut flora balance, provide diverse prebiotic fibers, and support different bacteria species. For example, in some embodiments, the probiotic blend comprises at least one of lactobacillus, bifidobacterium, Enterococcus faecium, Streptococcus thermophilus, or any combination thereof.

The probiotic blend can be present in an amount of 1 billion colony-forming units (CFU) to 2 billion CFU per serving, or any range or subrange between 1 billion CFU to 2 billion CFU as disclosed herein.

The grain-free companion supplement can comprise a herbal blend. The herbal blend can comprise at least one of a siliphos, a curcumin, an ashwagandha KSM-66, a kelp powder, or any combination thereof.

The siliphos can be present in an amount of 25 mg to 75 mg per serving, or any range or subrange between 25 mg to 75 mg.

The curcumin can be present in an amount of 25 mg to 75 mg per serving, or any range or subrange between 25 mg to 75 mg.

The ashwagandha KSM-66 can be present in an amount of 25 mg to 50 mg per serving, or any range or subrange between 25 mg to 50 mg.

The kelp powder can be present in an amount of 10 mg to 30 mg per serving, or any range or subrange between 10 mg to 30 mg.

The grain-free companion supplement can comprise at least one of a choline, a Coenzyme Q10, or any combination thereof.

Coenzyme Q10 can be present in the grain-free companion supplement in a precise dose. The Coenzyme Q10 can be present in an amount of 15 mg per serving.

The choline can be present in an amount of 25 mg to 75 mg per serving, or any range or subrange between 25 mg to 75 mg.

The grain-free companion supplement can be administered simultaneously (“co-administered”) with administering the grain-free KibbleX composition. In some embodiments, the grain-free companion supplement can be co-administered about 1 second to 5 minutes of administering the KibbleX composition, or any range or subrange between about 1 second and 5 minutes as disclosed herein. For example, in some embodiments, the grain-free companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the kibble composition.

Table 3 is an exemplary illustration of ingredients in an embodiment of a KibbleX Grain-Free All Life Stage Companion Supplement of the present disclosure developed in accordance with a method of the present disclosure based on the KibbleX Grain-Free All Life Stage base food having the guaranteed analysis nutritional information in Table 4.

TABLE 3
Supplement Facts
Serving Size 1 Soft Chew (4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 30 | IU | † |
| tocopherols) | | | |
| Skin & Coat Blend | | | |
| Zinc (as Zinc Carnosine) | 15 | mg | † |
| Omega-3 Fatty Acids | 200 | mg | † |
| (from Krill Oil) | | | |
| Biotin | 250 | mcg | † |
| Amino Acids | 220 | mg | |
| L-Carnitine | 150 | mg | † |
| Tryptophan | 70 | mg | † |
| Joint Support Blend | 780 | mg | |
| Glucosamine HCl | 275 | mg | † |
| Chondroitin Sulfate | 175 | mg | † |
| MSM (Methylsulfonylmethane) | 175 | mg | † |
| Collagen Peptides | 150 | mg | † |
| Manganese | 5 | mg | † |
| Digestive Support Blend | 320 | mg | |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 25 | mg | † |
| Bromelain | 20 | mg | † |
| Lipase | TBD | | † |
| Protease | TBD | | † |
| Amylase | TBD | | † |
| Probiotic Blend | 2 billion CFU | | † |
| Herbal Blend | 155 | mg | |
| Siliphos | 50 | mg | † |
| Curcumin (with Black | 50 | mg | † |
| Pepper Extract) | | | |
| Ashwagandha KSM-66 | 35 | mg | † |
| Kelp Powder | 20 | mg | † |
| Other Nutrients | | | |
| Choline | 50 | mg | † |
| Coenzyme Q10 | 15 | mg | † |

TABLE 4
Guaranteed Analysis - Grain Free All Life Stage Dog Base Food

| Crude Protein (min) | 30.0% | |
|---|---|---|
| Crude Fat (min) | 19.0% | |
| Crude Fiber (max) | 4.5% | |
| Moisture (max) | 10.0% | |
| Docosahexaenoic Acid (DHA) (min) | 0.10% | |
| Eicosapentaenoic Acid (EPA) (min) | 0.12% | |
| Calcium (min) | 1.4% | |
| Phosphorus (min) | 1.0% | |
| Vitamin A (min) | 24,000 | IU/kg |
| Vitamin E (min) | 350 | IU/kg |
| Ascorbic Acid (Vitamin C) (min) | 325 | mg/kg |
| Omega-3 Fatty Acids (min) | 0.75% | |
| Omega-6 Fatty Acids (min) | 3.75% | |
| Taurine (min) | 0.20% | |
| Total microorganisms (min) | 10,000,000 | CFU/lb |

In some embodiments, a method comprises administering between about 2 ounces to 6 ounces of a lamb meal and brown rice KibbleX composition to a dog, or any range of subrange between 2 ounces to 6 ounces.

In some embodiments, the administering comprises pouring between about 2 ounces to 6 ounces of a lamb meal and brown rice KibbleX composition to a dog. In some embodiments, the administering comprises scooping between about 2 ounces to 6 ounces of a lamb meal and brown rice kibble composition to a dog. In some embodiments, the administering comprises dispensing between about 2 ounces to 6 ounces of a lamb meal and brown rice kibble composition to a dog. In some embodiments, the administering comprises between about 2 ounces to 6 ounces of a lamb meal and brown rice kibble composition to a dog. In some embodiments, the administering comprises distributing between about 2 ounces to 6 ounces of a lamb meal and brown rice kibble composition to a dog. In some embodiments, administering comprises ingesting between about 2 ounces to 6 ounces of a lamb meal and brown rice kibble composition to a dog.

The lamb meal and brown rice KibbleX composition can have an improved protein composition compared to traditional kibble compositions. The lamb meal and brown rice KibbleX composition can comprise at least one of a lamb meal, an egg powder, a whiting meal, or any combination thereof.

The lamb meal and brown rice KibbleX composition can provide an alternative protein profile with lamb meal, egg powder, whiting meal, and balanced amino acids from other sources. The lamb meal and brown rice KibbleX composition can comprise a digestible carbohydrate matrix comprising at least one of brown rice, oat groats, pearled barley, or any combination thereof. Brown rice can be the primary carbohydrate source.

In some embodiments, the lamb meal and brown rice KibbleX composition can comprise about 15% to 35% lamb meal on a total weight basis of the lamb meal and brown rice KibbleX composition, or any range or subrange between about 15% and 35%. For example, in some embodiments, the lamb meal and brown rice KibbleX composition can comprise about 15% to 30%, 20% to 25%, 20% to 35%, 25% to 35%, 30% to 35%, 15% to 30%, 15% to 25%, or 15% to 20% lamb meal on a total weight basis of the lamb meal and brown rice KibbleX composition.

In some embodiments, the lamb meal and brown rice KibbleX composition comprises grains. In some embodiments, the grains comprises at least a brown rice, an oat groats, pearled barley, or any combination thereof.

In some embodiments, the lamb meal and brown rice KibbleX composition can comprise about 30% to 55% grains on a total weight basis of the KibbleX composition, or any range or subrange between 30% and 55%. For example, in some embodiments, the lamb meal and brown rice KibbleX composition can comprise about 35% to 50%, 35% to 45%, 30% to 50%, 30% to 45%, 30% to 40%, 30% to 35%, 35% to 55%, 40% to 55%, 45% to 55%, 50% to 55% grains on a total weight basis of the KibbleX composition.

In some embodiments, the lamb meal and brown rice KibbleX composition can comprise about 5% to 20% fat on a total weight basis of the lamb meal and brown rice KibbleX composition, or any range or subrange between 5% and 20%. For example, in some embodiments, the lamb meal and brown rice KibbleX composition can comprise about 10% to 15%, 5% to 15%, 5% to 10%, 10% to 20%, or 15% to 20% fat on a total weight basis of the lamb meal and brown rice KibbleX composition.

The lamb meal and brown rice KibbleX composition provides a proprietary fiber and prebiotic mix. In some embodiments, a beet pulp provides fermentable fiber. In some embodiments, the Diamond V supports digestive health.

In some embodiments, the method comprises administering between about 3 to 5 grams of a lamb meal and brown rice companion supplement. In some embodiments, the method comprises administering between about 3 to 5 grams of a lamb meal and brown rice companion supplement. In some embodiments, the administering comprises pouring between about 3 to 5 grams of lamb meal and brown rice companion supplement. In some embodiments, the administering comprises scooping between about 3 to 5 grams of a lamb meal and brown rice companion supplement. In some embodiments, the administering comprises dispensing between about 3 to 5 grams of a lamb meal and brown rice companion supplement. In some embodiments, the administering comprises between about 3 to 5 grams of a lamb meal and brown rice companion supplement. In some embodiments, the administering comprises distributing between about 3 to 5 grams of a lamb meal and brown rice companion supplement. In some embodiments, administering comprises ingesting between about 3 to 5 grams of a lamb meal and brown rice companion supplement.

The lamb meal and brown rice companion supplement can be for a grain-free for all life stages. For example, in some embodiments, the lamb meal and brown rice companion supplement is provided for puppies developing strong foundations. In some embodiments, the lamb meal and brown rice companion supplement is provided for adult dogs maintaining optimal health. In some embodiments, the lamb meal and brown rice companion supplement is provided for senior dogs needing extra support. In some embodiments, the lamb meal and brown rice companion supplement is provided for active dogs requiring joint support. In some embodiments, the lamb meal and brown rice companion supplement is provided for dogs experiencing stress or changes. In some embodiments, the lamb meal and brown rice companion supplement is provided for any dog needing comprehensive nutrition.

The lamb meal and brown rice companion supplement can be tailored to complement the lamb meal and brown rice kibble composition. For example, in some embodiments, the lamb meal and brown rice companion supplement can be a KibbleX Lamb Meal and Brown Rice All Life Stage Companion Supplement. The lamb meal and brown rice companion supplement can be in the form of a soft chew.

The lamb meal and brown rice companion supplement can optimize protein utilization, provide joint support, provide digestive support, improve skin and coat health, provide metabolic support, and provide immune system support in synergy with the grain-free kibble composition. The lamb meal and brown rice companion supplement can optimize protein utilization. It will be appreciated that any one or more of vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients disclosed herein can comprise the lamb meal and brown rice companion supplement. It will further be appreciated that the lamb meal and brown rice companion supplement can comprise the ingredients present in an amount similar, or the same as the companion supplement disclosed herein.

The lamb meal and brown rice companion supplement can deliver heat-sensitive nutrients at the nutrient's effective potency as disclosed herein. The lamb meal and brown rice companion supplement can have precise dosing of the nutrients to ensure optimal nutrient levels.

The lamb meal and brown rice companion supplement can comprise at least one of Vitamin C, a Vitamin E, or any combination thereof. The Vitamin C can be present in an amount of 30 mg to 70 mg per serving, or any range or subrange between 30 milligrams mg and 70 mg as disclosed herein.

The Vitamin E can be present in an amount of 20 IU to 60 IU per serving, or any range or subrange between 20 IU and 60 IU as disclosed herein.

The lamb meal and brown rice companion supplement can comprise a skin and coat blend. The skin and coat blend comprises at least one of a zinc, DHA, a biotin, or any combination thereof. The lamb meal and brown rice companion supplement can elevate skin and coat support without requiring precise dosing of various nutrients.

Zinc can be present in the lamb meal and brown rice companion supplement in a precise dose. The zinc can be present in an amount of 20 mg per serving.

Biotin can be present in an amount of 200 micrograms (mcg) to 300 mcg per serving, or any range or subrange between 200 mcg and 300 mcg as disclosed herein.

Omega-3 Fatty Acids can be present in an amount of 250 mg to 350 mg per serving, or any range or subrange between 250 mg and 350 mg. In some embodiments, the Omega-3 Fatty Acids can be present in an amount of 260 mg to 340 mg, 270 mg to 330 mg, 280 mg to 320 mg, or 290 mg to 310 mg per serving. In some embodiments, the Omega-3 Fatty Acids can be present in an amount of 260 mg to 350 mg, 270 mg to 350 mg, 280 mg to 350 mg, 290 mg to 350 mg, 300 mg to 350 mg, 310 mg to 350 mg, 320 mg to 350 mg, 330 mg to 350 mg, or 340 mg to 350 mg per serving. In some embodiments, the Omega-3 Fatty Acids can be present in an amount of 250 mg to 340 mg, 250 mg to 330 mg, 250 mg to 320 mg, 250 mg to 310 mg, 250 mg to 300 mg, 250 mg to 290 mg, 250 mg to 280 mg, 250 mg to 270 mg, 250 mg to 260 mg per serving.

The lamb meal and brown rice companion supplement can comprise amino acids. The amino acids comprises at least one of a L-Carnitine, a tryptophan, or any combination thereof.

Tryptophan can be present in an amount of 20 mg to 80 mg per serving, or any range or subrange between 20 mg and 80 mg. For example, in some embodiments, tryptophan can be present in an amount of 30 mg to 70 mg, 40 mg to 60 mg, 30 mg to 60 mg, 30 mg to 50 mg, 30 mg to 40 mg, 30 mg to 80 mg, 40 mg to 80 mg, 50 mg to 80 mg, 60 mg to 80 mg, or 70 mg to 80 mg per serving.

L-Carnitine can be present in the lamb meal and brown rice companion supplement in a precise dose. The L-Carnitine can be present in an amount of 100 mg per serving.

The lamb meal and brown rice companion supplement can comprise a joint support blend. The joint support blend comprises a glucosamine HCl, a chondroitin sulfate, a MSM, collagen peptides, a manganese, or any combination thereof.

Glucosamine HCl can be present in an amount of 250 mg to 300 mg per serving, or any range or subrange between 250 mg and 300 mg as disclosed herein.

MSM can be present in the lamb meal and brown rice companion supplement in a precise dose. The MSM can be present in an amount of 175 mg per serving.

Chondroitin sulfate can be present in an amount of 150 mg to 200 mg per serving, or any range or subrange between 150 mg and 200 mg as disclosed herein.

Collagen peptides can be present in an amount of 100 mg to 200 mg per serving, or any range or subrange between 100 mg and 200 mg as disclosed herein.

Manganese can be present in an amount of 1 mg to 10 mg per serving, or any range or subrange between 1 mg and 10 mg.

The lamb meal and brown rice companion supplement can comprise a digestive support blend. The digestive support blend can comprise at least one of an inulin, a fructooligosaccharides, a mannanoligosaccharides, Beta-glucans, abinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend.

Inulin can be present in an amount of 50 mg to 150 mg per serving, or any range or subrange between 50 mg and 150 mg as disclosed herein.

Fructooligosaccharides can be present in an amount of 50 mg to 100 mg per serving, or any range or subrange between 50 mg and 100 mg as disclosed herein.

Mannanoligosaccharides can be present in an amount of 25 mg to 75 mg per serving, or any range or subrange between 25 mg and 75 mg as disclosed herein.

Beta-glucans can be present in an amount of 15 mg to 35 mg per serving, or any range or subrange between 15 mg and 35 mg as disclosed herein.

Arabinogalactans can be present in an amount of 15 mg to 35 mg per serving, or any range or subrange between 15 mg and 35 mg as disclosed herein.

Bromelain can be present in an amount of 20 mg to 40 mg per serving, or any range or subrange between 20 mg and 40 mg. For example, in some embodiments, bromelain can be present in an amount of 25 mg to 35 mg, 20 mg to 35 mg, 20 mg to 30 mg, 20 mg to 25 mg, 25 mg to 40 mg, 30 mg to 40 mg, or 35 mg to 40 mg per serving.

Ginger root extract can be present in an amount of 25 mg to 45 mg per serving, or any range or subrange between 25 mg and 45 mg. In some embodiments, ginger root extract can be present in an amount of 30 mg to 40 mg, 25 mg to 40 mg, 25 mg to 35 mg, 25 mg to 30 mg, 30 mg to 45 mg, 35 mg to 45 mg, or 40 mg to 45 mg per serving.

The lamb meal and brown rice companion supplement can comprise a probiotic blend. For example, in some embodiments, the probiotic blend comprises at least one of lactobacillus, bifidobacterium, Enterococcus faecium, Streptococcus thermophilus, or any combination thereof.

The probiotic blend can be present in an amount of 1 billion CFU to 2 billion CFU per serving, or any range or subrange between 1 billion CFU to 2 billion CFU as disclosed herein.

The lamb meal and brown rice companion supplement can comprise a herbal blend. The herbal blend can comprise at least one of a siliphos, a curcumin, an ashwagandha KSM-66, a kelp powder, or any combination thereof.

The siliphos can be present in an amount of 25 mg to 75 mg per serving, or any range or subrange between 25 mg to 75 mg. The curcumin can be present in an amount of 25 mg to 75 mg per serving, or any range or subrange between 25 mg to 75 mg.

The ashwagandha KSM-66 can be present in an amount of 25 mg to 50 mg per serving, or any range or subrange between 25 mg to 50 mg.

The kelp powder can be present in an amount of 15 mg to 35 mg per serving, or any range or subrange between 10 mg to 30 mg. In some embodiments, the kelp powder can be present in an amount of 15 mg to 25 mg, 10 mg to 25 mg, 10 mg to 20 mg, 10 mg to 15 mg, 15 mg to 30 mg, 15 mg to 25 mg, or 15 mg to 20 mg per serving.

The lamb meal and brown rice companion supplement can comprise at least one of a choline, a Coenzyme Q10, or any combination thereof. Coenzyme Q10 can be present in the companion supplement in a precise dose. The Coenzyme Q10 can be present in an amount of 15 mg per serving.

The choline can be present in an amount of 25 mg to 75 mg per serving, or any range or subrange between 25 mg to 75 mg.

The lamb meal and brown rice companion supplement can be administered simultaneously (“co-administered”) with administering the KibbleX composition. In some embodiments, the lamb meal and brown rice companion supplement can be co-administered about 1 second to 5 minutes of administering the KibbleX composition, or any range or subrange between about 1 second and 5 minutes. For example, in some embodiments, the lamb meal and brown rice companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the kibble composition.

Table 5 is an exemplary illustration of ingredients in aa embodiment of a KibbleX Lamb Meal and Brown Rice All Life Stage Companion Supplement of the present disclosure developed in accordance with a method of the present disclosure based on the KibbleX Lamb Meal and Brown Rice base food having the guaranteed analysis nutritional information listed in Table 6.

TABLE 5
Supplement Facts
Serving Size 1 Soft Chew (4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 40 | IU | † |
| tocopherols) | | | |
| Skin & Coat Blend | | | |
| Zinc (as Zinc Carnosine) | 20 | mg | † |
| Omega-3 Fatty Acids | 300 | mg | † |
| (from Krill Oil) | | | |
| Biotin | 250 | mcg | † |
| Amino Acids | 150 | mg | |
| L-Carnitine | 100 | mg | † |
| Tryptophan | 50 | mg | † |
| Joint Support Blend | 780 | mg | |
| Glucosamine HCl | 275 | mg | † |
| Chondroitin Sulfate | 175 | mg | † |
| MSM (Methylsulfonylmethane) | 175 | mg | † |
| Collagen Peptides | 150 | mg | † |
| Manganese | 5 | mg | † |
| Digestive Support Blend | 340 | mg | |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 35 | mg | † |
| Bromelain | 30 | mg | † |
| Lipase | TBD | | † |
| Protease | TBD | | † |
| Amylase | TBD | | † |
| Probiotic Blend | 2 billion CFU | | † |
| Herbal Blend | 160 | mg | |
| Siliphos | 50 | mg | † |
| Curcumin (with Black | 50 | mg | † |
| Pepper Extract) | | | |
| Ashwagandha KSM-66 | 35 | mg | † |
| Kelp Powder | 25 | mg | † |
| Other Nutrients | | | |
| Choline | 50 | mg | † |
| Coenzyme Q10 | 15 | mg | † |

TABLE 6
Guaranteed Analysis - Lamb Meal
& Brown Rice Recipe Base Food

| Crude Protein (min) | 26.0% | |
|---|---|---|
| Crude Fat (min) | 16.0% | |
| Crude Fiber (max) | 5.0% | |
| Moisture (max) | 10.0% | |
| Docosahexaenoic Acid (DHA) (min) | 0.08% | |
| Eicosapentaenoic Acid (EPA) (min) | 0.10% | |
| Calcium (min) | 1.4% | |
| Phosphorus (min) | 1.0% | |
| Vitamin A (min) | 24,000 | IU/kg |
| Vitamin E (min) | 350 | IU/kg |
| Ascorbic Acid (Vitamin C) (min) | 325 | mg/kg |
| Omega-3 Fatty Acids (min) | 0.75% | |
| Omega-6 Fatty Acids (min) | 3.50% | |
| Taurine (min) | 0.12% | |
| Total microorganisms (min) | 10,000,000 | CFU/lb |

In some embodiments, a method comprises administering between about 2 ounces to 6 ounces of a small/medium breed puppy KibbleX composition to a dog, or any range of subrange between 2 ounces to 6 ounces.

In some embodiments, the administering comprises pouring between about 2 ounces to 6 ounces of a small/medium breed puppy kibble composition to a dog. In some embodiments, the administering comprises scooping between about 2 ounces to 6 ounces of a small/medium breed puppy KibbleX composition to a dog. In some embodiments, the administering comprises dispensing between about 2 ounces to 6 ounces of a small/medium breed puppy KibbleX composition to a dog. In some embodiments, the administering comprises between about 2 ounces to 6 ounces of a small/medium breed puppy KibbleX composition to a dog. In some embodiments, the administering comprises distributing between about 2 ounces to 6 ounces of a small/medium breed puppy KibbleX composition to a dog. In some embodiments, administering comprises ingesting between about 2 ounces to 6 ounces of a small/medium breed puppy KibbleX composition to a dog.

In some embodiments, the small/medium breed puppy KibbleX composition can comprise protein from a protein source. In some embodiments, the protein source comprises at least one of a chicken meal, a turkey meal, lamb meal, fish meal, or any combination thereof. In some embodiments, the fish meal can comprise salmon.

The small/medium breed puppy KibbleX composition can have an improved protein composition compared to traditional kibble compositions. In some embodiments, the small/medium breed puppy KibbleX composition can comprise about 20% to 35% protein from a protein source on a total weight basis of the small/medium breed puppy KibbleX composition, or any range or subrange between about 20% and 35%. For example, in some embodiments, the KibbleX composition can comprise about 25% to 30%, 20% to 30%, 20% to 25%, 25% to 35%, or 30% to 35% protein from a protein source on a total weight basis of the KibbleX composition.

In some embodiments, the small/medium breed puppy KibbleX composition comprises grains. In some embodiments, the grains comprises at least a brown rice, an oat groats, sweet potato, or any combination thereof.

In some embodiments, the small/medium breed puppy KibbleX composition can comprise about 30% to 55% grains on a total weight basis of the small/medium breed puppy KibbleX composition, or any range or subrange between 30% and 55%. For example, in some embodiments, the small/medium breed puppy KibbleX composition can comprise about 35% to 50%, 35% to 45%, 30% to 50%, 30% to 45%, 30% to 40%, 30% to 35%, 35% to 55%, 40% to 55%, 45% to 55%, 50% to 55% grains on a total weight basis of the small/medium breed puppy KibbleX composition.

In some embodiments, the small/medium breed puppy KibbleX composition can comprise about 5% to 25% chicken fat on a total weight basis of the small/medium breed puppy KibbleX composition, or any range or subrange between 5% and 25%. For example, in some embodiments, the small/medium breed puppy KibbleX composition can comprise about 10% to 20%, 5% to 20%, 5% to 15%, 5% to 10%, 10% to 25%, 15% to 25%, or 20% to 25% chicken fat on a total weight basis of the small/medium breed puppy KibbleX composition.

The small/medium breed puppy KibbleX composition can provide growth support complex for a growing dog. The small/medium breed puppy KibbleX composition can comprise provide for increased energy and protein content. Protein sources can comprise the chicken meal and egg powder.

The small/medium breed puppy KibbleX composition provides a proprietary fiber and prebiotic mix.

In some embodiments, the method comprises administering between about 3 to 5 grams of small/medium breed puppy companion supplement. In some embodiments, the method comprises administering between about 3 to 5 grams of a small/medium breed puppy companion supplement. In some embodiments, the administering comprises pouring between about 3 to 5 grams of a small/medium breed puppy companion supplement. In some embodiments, the administering comprises scooping between about 3 to 5 grams of a small/medium breed puppy companion supplement. In some embodiments, the administering comprises dispensing between about 3 to 5 grams of a small/medium breed puppy companion supplement. In some embodiments, the administering comprises between about 3 to 5 grams of a small/medium breed puppy companion supplement. In some embodiments, the administering comprises distributing between about 3 to 5 grams of a small/medium breed puppy companion supplement. In some embodiments, administering comprises ingesting between about 3 to 5 grams of a small/medium breed puppy companion supplement.

In some embodiments, the small/medium breed puppy companion supplement is provided for puppies developing strong foundations.

The small/medium breed puppy companion supplement can be tailored to complement the small/medium breed puppy KibbleX composition. The small/medium breed puppy companion supplement can be in the form of a soft chew.

The small/medium breed puppy companion supplement can optimize protein utilization, provide joint development support, provide digestive support, improve skin and coat health, provide improved nutrient absorption, provide brain development support, and provide immune system support in synergy with the small/medium breed puppy kibble composition. The small/medium breed puppy companion supplement can optimize protein utilization. It will be appreciated that any one or more of vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients disclosed herein can comprise the small/medium breed puppy companion supplement. It will further be appreciated that the small/medium breed puppy companion supplement can comprise the ingredients present in an amount similar, or the same as the companion supplement disclosed herein.

The small/medium breed puppy companion supplement can deliver heat-sensitive nutrients at the nutrient's effective potency as disclosed herein. The small/medium breed puppy companion supplement can have precise dosing of the nutrients to ensure optimal nutrient levels.

The small/medium breed puppy companion supplement can comprise at least one of Vitamin C, a Vitamin E, or any combination thereof. The Vitamin C can be present in an amount of 30 mg to 70 mg per serving, or any range or subrange between 30 milligrams mg and 70 mg as disclosed herein.

The Vitamin E can be present in an amount of 20 IU to 60 IU per serving, or any range or subrange between 20 IU and 60 IU.

The small/medium breed puppy companion supplement can comprise a skin and coat blend. The skin and coat blend comprises at least one of a zinc, DHA, a biotin, or any combination thereof. The small/medium breed puppy companion supplement can elevate skin and coat support without requiring precise dosing of various nutrients.

Zinc can be present in the small/medium breed puppy companion supplement in a precise dose. The zinc can be present in an amount of 20 mg per serving.

Biotin can be present in an amount of 200 micrograms (mcg) to 300 mcg per serving, or any range or subrange between 200 mcg and 300 mcg as disclosed herein.

DHA can be present in an amount of 50 mg to 150 mg per serving, or any range or subrange between 50 mg and 150 mg.

The small/medium breed puppy companion supplement can comprise amino acids. The amino acids comprises at least one of a L-Carnitine, a tryptophan, or any combination thereof.

Tryptophan can be present in an amount of 125 mg to 175 mg per serving, or any range or subrange between 125 mg and 175 mg.

The small/medium puppy companion supplement can comprise L-Carnitine. L-Carnitine can be present in the companion supplement in a precise dose. The L-Carnitine can be present in an amount of 125 mg per serving.

The small/medium puppy companion supplement can comprise a joint support blend. The joint support blend comprises a glucosamine HCl, a chondroitin sulfate, a methylsulfonylmethane (MSM), collagen peptides, a manganese, or any combination thereof.

Glucosamine HCl can be present in the small/medium breed puppy companion supplement in a precise ratio for optimal absorption compared to sulfate forms. Glucosamine HCl can be present in an amount of 175 mg to 225 mg per serving, or any range or subrange between 175 mg and 225 mg. For example, in some embodiments, glucosamine HCl can be present in an amount of 180 mg to 220 mg, 185 mg to 215 mg, 190 mg to 210 mg, or 195 mg to 205 mg per serving. In some embodiments, glucosamine HCl can be present in an amount of 180 mg to 225 mg, 185 mg to 225 mg, 190 mg to 225 mg, 195 mg to 225 mg, 200 mg to 225 mg, 205 mg to 225 mg, 210 mg to 225 mg, 215 mg to 225 mg, or 220 mg to 225 mg per serving. In some embodiments, glucosamine HCl can be present in an amount of 175 mg to 220 mg, 175 mg to 215 mg, 175 mg to 210 mg, 175 mg to 205 mg, 175 mg to 200 mg, 175 mg to 195 mg, 175 mg to 190 mg, 175 mg to 185 mg, or 175 mg to 180 mg per serving.

MSM can be present in the small/medium breed puppy companion supplement in a precise dose. The MSM can be present in an amount of 150 mg per serving.

Chondroitin sulfate can work synergistically with glucosamine HCl to maintain cartilage structure and joint fluid viscosity. Chondroitin sulfate can be present in the companion supplement in a precise ratio for optimal absorption. Chondroitin sulfate can be present in an amount of 100 mg to 200 mg per serving, or any range or subrange between 125 mg and 175 mg. For example, in some embodiments, chondroitin sulfate can be present in an amount of 110 mg to 190 mg, 120 mg to 180 mg, 130 mg to 170 mg, or 140 mg to 160 mg per serving. In some embodiments, chondroitin sulfate can be present in an amount of 110 mg to 200 mg, 120 mg to 200 mg, 130 mg to 200 mg, 140 mg to 200 mg, 150 mg to 200 mg, 160 mg to 200 mg, 170 mg to 200 mg, 180 mg to 200 mg, or 190 mg to 200 mg per serving. In some embodiments, chondroitin sulfate can be present in an amount of 100 mg to 190 mg, 100 mg to 180 mg, 100 mg to 170 mg, 100 mg to 160 mg, 100 mg to 150 mg, 100 mg to 140 mg, 100 mg to 130 mg, 100 mg to 120 mg, or 100 mg to 110 mg per serving.

Collagen peptides can be present in an amount of 50 mg to 150 mg per serving, or any range or subrange between 50 mg and 150 mg. For example, in some embodiments, collagen peptides can be present in an amount of 60 mg to 140 mg, 70 mg to 130 mg, 80 mg to 120 mg, or 90 mg to 110 mg per serving. In some embodiments, collagen peptides can be present in an amount of 50 mg to 140 mg, 50 mg to 130 mg, 50 mg to 120 mg, 50 mg to 110 mg, 50 mg to 100 mg, 50 mg to 90 mg, 50 mg to 80 mg, 50 mg to 70 mg, or 50 mg to 60 mg per serving. In some embodiments, collagen peptides can be present in an amount of 60 mg to 150 mg, 70 mg to 150 mg, 80 mg to 150 mg, 90 mg to 150 mg, 100 mg to 150 mg, 110 mg to 150 mg, 120 mg to 150 mg, 130 mg to 150 mg, or 140 mg to 150 mg per serving.

Manganese can be present in an amount of 1 mg to 10 mg per serving, or any range or subrange between 1 mg and 10 mg.

The small/medium breed puppy companion supplement can comprise a digestive support blend. The digestive support blend can comprise at least one of an inulin, a fructooligosaccharides, a mannanoligosaccharides, Beta-glucans, arabinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend. The prebiotic blend can comprise the inulin, the fructooligosaccharides the mannanoligosaccharides, the Beta-glucans, and the arabinogalactans.

Inulin can provide a long-chain prebiotic fiber supporting sustained fermentation. Inulin can be present in an amount of 50 mg to 150 mg per serving, or any range or subrange between 50 mg and 150 mg.

Fructooligosaccharides can be present in an amount of 50 mg to 100 mg per serving, or any range or subrange between 50 mg and 100 mg.

Mannanoligosaccharides can be present in an amount of 25 mg to 75 mg per serving, or any range or subrange between 25 mg and 75 mg.

Beta-glucans can provide immune-modulating prebiotics. Beta-glucans can be present in an amount of 15 mg to 35 mg per serving, or any range or subrange between 25 mg and 75 mg.

Arabinogalactans can provide a plant-based prebiotic to support bacteria growth. Arabinogalactans can be present in an amount of 15 mg to 35 mg per serving, or any range or subrange between 15 mg and 35 mg.

Bromelain is a protein-digesting enzyme. Bromelain can be present in an amount of 15 mg to 35 mg per serving, or any range or subrange between 10 mg and 30 mg. For example, in some embodiments, bromelain can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 mg per serving.

Ginger root extract can provide support for digestive comfort and motility. Ginger root extract can be present in an amount of 15 mg to 35 mg per serving, or any range or subrange between 15 mg and 35 mg.

The small/medium breed puppy companion supplement can comprise a probiotic blend. For example, in some embodiments, the probiotic blend comprises at least one of lactobacillus, bifidobacterium, Enterococcus faecium, Streptococcus thermophilus, or any combination thereof.

The probiotic blend can be present in an amount of 2 billion colony-forming units (CFU) to 4 billion CFU per serving, or any range or subrange between 2 billion CFU to 4 billion CFU. For example, in some embodiments, the probiotic blend can be present in an amount of 2.5 billion CFU to 3.5 billion CFU, 2 billion CFU to 3.5 billion CFU, 2 billion CFU to 3 billion CFU, 2 billion CFU to 2.5 billion CFU, 2.5 billion CFU to 4 billion CFU, 3 billion CFU to 4 billion CFU, or 3.5 billion CFU to 4 billion CFU per serving.

The small/medium breed puppy companion supplement can comprise a herbal blend. The herbal blend can comprise at least one of a siliphos, a chamomile extract, a fennel seed extract, a kelp powder, or any combination thereof.

The siliphos can be present in an amount of 10 mg to 50 mg per serving, or any range or subrange between 10 mg and 50 mg. For example, in some embodiments, siliphos can be present in an amount of 15 mg to 45 mg, 20 mg to 40 mg, or 25 mg to 35 mg per serving. In some embodiments, siliphos can be present in an amount of 15 mg to 50 mg, 20 mg to 50 mg, 25 mg to 50 mg, 30 mg to 50 mg, 35 mg to 50 mg, 40 mg to 50 mg, or 45 mg to 50 mg per serving. In some embodiments, siliphos can be present in an amount of 10 mg to 45 mg, 10 mg to 40 mg, 10 mg to 35 mg, 10 mg to 30 mg, 10 mg to 25 mg, 10 mg to 20 mg, or 10 mg to 15 mg per serving.

The chamomile extract can be present in an amount of 30 mg to 70 mg per serving, or any range or subrange between 30 mg and 70 mg. For example, in some embodiments, chamomile extract can be present in amount of 35 mg to 65 mg, 40 mg to 60 mg, or 45 mg to 55 mg per serving. In some embodiments, chamomile extract can be present in amount of 35 mg to 70 mg, 40 mg to 70 mg, or 45 mg to 70 mg, 50 mg to 70 mg, 55 mg to 70 mg, 60 mg to 70 mg, or 65 mg to 70 mg per serving. In some embodiments, chamomile extract can be present in amount of 30 mg to 65 mg, 30 mg to 60 mg, 30 mg to 55 mg, 30 mg to 50 mg, 30 mg to 45 mg, 30 mg to 40 mg, or 30 mg to 35 mg per serving.

The fennel seed extract can be present in an amount of 15 mg to 35 mg per serving, or any range or subrange between 15 mg and 35 mg. For example, in some embodiments, fennel seed extract can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 per serving.

The kelp powder can be present in an amount of 15 mg to 35 mg per serving, or any range or subrange between 15 mg to 35 mg. For example, in some embodiments, kelp powder can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 per serving.

The small/medium breed puppy companion supplement can comprise a growth support blend. The herbal blend can comprise at least one of a colostrum, nucleotides, egg protein peptides, choline, or any combination thereof.

The colostrum can be present in an amount of 50 mg to 150 mg per serving, or any range or subrange between 50 mg to 150 mg. For example, in some embodiments, colostrum can be present in an amount of 60 mg to 140 mg, 70 mg to 130 mg, 80 mg to 120 mg, or 90 mg to 110 mg per serving. In some embodiments, colostrum can be present in an amount of 50 mg to 140 mg, 50 mg to 130 mg, 50 mg to 120 mg, 50 mg to 110 mg, 50 mg to 100 mg, 50 mg to 90 mg, 50 mg to 80 mg, 50 mg to 70 mg, or 50 mg to 60 mg per serving. In some embodiments, colostrum can be present in an amount of 60 mg to 150 mg, 70 mg to 150 mg, 80 mg to 150 mg, 90 mg to 150 mg, 100 mg to 150 mg, 110 mg to 150 mg, 120 mg to 150 mg, 130 mg to 150 mg, or 140 mg to 150 mg per serving.

The nucleotides can be present in an amount of 50 mg to 100 mg per serving, or any range or subrange between 50 mg to 100 mg. For example, in some embodiments, nucleotides can be present in an amount of 55 mg to 95 mg, 60 mg to 90 mg, 65 mg to 85 mg, or 70 mg to 80 mg per serving. In some embodiments, nucleotides can be present in an amount of 55 mg to 100 mg, 60 mg to 100 mg, 65 mg to 100 mg, 70 mg to 100 mg, 75 mg to 100 mg, 80 mg to 100 mg, 85 mg to 100 mg, 90 mg to 100 mg, or 95 mg to 100 mg per serving. In some embodiments, nucleotides can be present in an amount of 50 mg to 95 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg per serving.

The egg protein peptides can be present in an amount of 50 mg to 100 mg per serving, or any range or subrange between 50 mg to 100 mg. For example, in some embodiments, egg protein peptides can be present in an amount of 55 mg to 95 mg, 60 mg to 90 mg, 65 mg to 85 mg, or 70 mg to 80 mg per serving. In some embodiments, egg protein peptides can be present in an amount of 55 mg to 100 mg, 60 mg to 100 mg, 65 mg to 100 mg, 70 mg to 100 mg, 75 mg to 100 mg, 80 mg to 100 mg, 85 mg to 100 mg, 90 mg to 100 mg, or 95 mg to 100 mg per serving. In some embodiments, egg protein peptides can be present in an amount of 50 mg to 95 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg per serving.

The choline can be present in an amount of 25 mg to 75 mg per serving, or any range or subrange between 25 mg to 75 mg. For example, in some embodiments, choline can be present in an amount of 30 mg to 70 mg, 35 mg to 65 mg, 40 mg to 60 mg, or 45 mg to 55 mg per serving. In some embodiments, choline can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, choline can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving.

The small/medium breed puppy companion supplement can comprise a Coenzyme Q10. Coenzyme Q10 can be present in the companion supplement in a precise dose. The Coenzyme Q10 can be present in an amount of 15 mg per serving.

The small/medium breed puppy companion supplement can be administered simultaneously (“co-administered”) with administering the small/medium breed puppy KibbleX composition. In some embodiments, the companion supplement can be co-administered about 1 second to 5 minutes of administering the small/medium breed puppy KibbleX composition, or any range or subrange between about 1 second and 5 minutes as disclosed herein. For example, in some embodiments, the small/medium breed puppy companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the kibble composition.

Table 7 is an exemplary illustration of ingredients in an embodiment of a KibbleX Small/Medium Breed Puppy Companion Supplement of the present disclosure developed in accordance with a method of the present disclosure based on the KibbleX Small/Medium Breed Puppy base food having the guaranteed analysis nutritional information listed in Table 8.

TABLE 7
Supplement Facts
Serving Size 1 Soft Chew (4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 35 | IU | † |
| tocopherols) | | | |
| Skin & Coat Blend | | | |
| Zinc (as Zinc | 20 | mg | † |
| Carnosine) | | | |
| DHA (from Algae) | 100 | mg | † |
| Biotin | 250 | mcg | † |
| Amino Acids | | | |
| L-Carnitine | 125 | mg | † |
| Tryptophan | 150 | mg | † |
| Joint Development Blend | 605 | mg | |
| Glucosamine HCl | 200 | mg | † |
| Chondroitin Sulfate | 150 | mg | † |
| MSM | 150 | mg | † |
| Collagen Peptides | 100 | mg | † |
| Manganese | 5 | mg | † |
| Digestive Support Blend | 275 | mg | |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 25 | mg | † |
| Bromelain | 25 | mg | † |
| Lipase | TBD | | † |
| Protease | TBD | | † |
| Amylase | TBD | | |
| Probiotic Blend | 3 billion CFU | | † |
| Growth Support Blend | 300 | mg | |
| Colostrum | 100 | mg | † |
| Nucleotides | 75 | mg | † |
| Egg Protein Peptides | 75 | mg | † |
| Choline | 50 | mg | † |
| Herbal Blend | 180 | mg | |
| Siliphos | 30 | mg | † |
| Chamomile Extract | 50 | mg | † |
| Fennel Seed Extract | 25 | mg | † |
| Kelp Powder | 25 | mg | † |
| Other Nutrients | | | |
| Coenzyme Q10 | 15 | mg | † |

TABLE 8
Guaranteed Analysis - Small/Medium Breed Puppy Base Food

| Crude Protein (min) | 27.0% | |
|---|---|---|
| Crude Fat (min) | 17.0% | |
| Crude Fiber (max) | 4.5% | |
| Moisture (max) | 10.0% | |
| Docosahexaenoic Acid (DHA) (min) | 0.15% | |
| Eicosapentaenoic Acid (EPA) (min) | 0.17% | |
| Calcium (min) | 1.4% | |
| Phosphorus (min) | 1.0% | |
| Vitamin A (min) | 24,000 | IU/kg |
| Vitamin E (min) | 350 | IU/kg |
| Ascorbic Acid (Vitamin C) (min) | 140 | mg/kg |
| Omega-3 Fatty Acids (min) | 0.70% | |
| Omega-6 Fatty Acids (min) | 3.50% | |
| Taurine (min) | 0.20% | |
| Total microorganisms (min) | 10,000,000 | CFU/lb |

In some embodiments, a method comprises administering between about 2 ounces to 6 ounces of a large breed puppy KibbleX composition to a dog, or any range of subrange between about 2 ounces and 6 ounces.

In some embodiments, the administering comprises pouring between about 2 ounces and 6 ounces of a large breed puppy KibbleX composition to a dog. In some embodiments, the administering comprises scooping between about 2 ounces and 6 ounces of a large breed puppy KibbleX composition to a dog. In some embodiments, the administering comprises dispensing between about 2 ounces and 6 ounces of a large breed puppy KibbleX composition to a dog. In some embodiments, the administering comprises between about 2 ounces and 6 ounces of a large breed puppy KibbleX composition to a dog. In some embodiments, the administering comprises distributing between about 2 ounces and 6 ounces of a large breed puppy KibbleX composition to a dog. In some embodiments, administering comprises ingesting between about 2 ounces and 6 ounces of a large breed puppy KibbleX composition to a dog.

The large breed puppy KibbleX composition can have an improved protein composition compared to traditional kibble compositions. In some embodiments, the large breed puppy KibbleX composition can comprise protein from a protein source. In some embodiments, the protein source comprises at least one of a chicken meal, a turkey meal, lamb meal, fish meal, or any combination thereof. In some embodiments, the fish meal can comprise salmon.

In some embodiments, the large breed puppy KibbleX composition can comprise about 15% to 35% protein from a protein source on a total weight basis of the KibbleX composition, or any range or subrange between about 15% and 35%. For example, in some embodiments, the large breed puppy KibbleX composition can comprise about 20% to 30%, 15% to 30%, 15% to 25%, 15% to 20%, 20% to 35%, 25% to 35%, or 30% to 35% protein from a protein source on a total weight basis of the KibbleX composition.

In some embodiments, the large breed puppy KibbleX composition comprises grains. In some embodiments, the grains comprises at least a brown rice, an oat groats, sweet potato, white potato, or any combination thereof.

In some embodiments, the large breed puppy KibbleX composition can comprise about 35% to 55% grains on a total weight basis of the KibbleX composition, or any range or subrange between 35% and 55%. For example, in some embodiments, the large breed puppy KibbleX composition can comprise about 40% to 50%, 35% to 50%, 35% to 45%, 35% to 40%, 40% to 55%, 45% to 55%, 50% to 55% grains on a total weight basis of the KibbleX composition.

In some embodiments, the large breed puppy KibbleX composition can comprise about 5% to 20% chicken fat on a total weight basis of the KibbleX composition, or any range or subrange between 5% and 20%. For example, in some embodiments, the large breed puppy KibbleX composition can comprise about 10% to 15%, 5% to 15%, 5% to 10%, 10% to 20%, or 15% to 20% chicken fat on a total weight basis of the KibbleX composition.

The large breed puppy KibbleX composition can provide growth support complex for a growing dog. The large breed puppy KibbleX composition can comprise provide for controlled protein and energy. In some embodiments, the large breed puppy KibbleX composition can provide moderate protein content to support steady growth. In some embodiments, the large breed puppy KibbleX composition can provide controlled caloric density to prevent rapid weight gain.

The large breed puppy KibbleX composition provides a proprietary fiber and prebiotic mix.

In some embodiments, the method comprises administering between about 3 to 5 grams of a large breed puppy companion supplement. In some embodiments, the method comprises administering between about 3 to 5 grams of a large breed puppy companion supplement. In some embodiments, the administering comprises pouring between about 3 to 5 grams of a large breed puppy companion supplement. In some embodiments, the administering comprises scooping between about 3 to 5 grams of a large breed puppy companion supplement. In some embodiments, the administering comprises dispensing between about 3 to 5 grams of a large breed puppy companion supplement. In some embodiments, the administering comprises between about 3 to 5 grams of a large breed puppy companion supplement. In some embodiments, the administering comprises distributing between about 3 to 5 grams of a large breed puppy companion supplement. In some embodiments, administering comprises ingesting between about 3 to 5 grams of a large breed puppy companion supplement.

In some embodiments, the large breed puppy companion supplement is provided for puppies developing strong foundations.

The large breed puppy companion supplement can be tailored to complement the large breed puppy KibbleX composition. For example, in some embodiments, the large breed puppy companion supplement can be a Large Breed Puppy Companion Supplement. The large breed puppy companion supplement can be in the form of a soft chew.

The large breed puppy companion supplement can optimize protein utilization, provide joint development support, provide digestive support, improve skin and coat health, provide improved nutrient absorption, provide brain development support, and provide immune system support in synergy with the large breed puppy kibble composition. The large breed puppy companion supplement can optimize protein utilization. It will be appreciated that any one or more of vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients disclosed herein can comprise the large breed puppy companion supplement. It will further be appreciated that the large breed puppy companion supplement can comprise the ingredients present in an amount similar, or the same as the companion supplement disclosed herein.

The large breed puppy companion supplement can deliver heat-sensitive nutrients at the nutrient's effective potency as disclosed herein. The large breed puppy companion supplement can have precise dosing of the nutrients to ensure optimal nutrient levels.

The large breed puppy companion supplement can comprise at least one of Vitamin C, a Vitamin E, or any combination thereof. The Vitamin C can be present in an amount of 30 mg to 70 mg per serving, or any range or subrange between 30 milligrams mg and 70 mg as disclosed herein.

The Vitamin E can be present in an amount of 20 IU to 60 IU per serving, or any range or subrange between 20 IU and 60 IU.

The large breed puppy companion supplement can comprise a skin and coat blend. The skin and coat blend comprises at least one of a zinc, DHA, a biotin, or any combination thereof. The small/medium breed puppy companion supplement can elevate skin and coat support without requiring precise dosing of various nutrients.

Zinc can be present in the large breed puppy companion supplement in a precise dose. The zinc can be present in an amount of 15 mg per serving.

Biotin can be present in an amount of 200 micrograms (mcg) to 300 mcg per serving, or any range or subrange between 200 mcg and 300 mcg as disclosed herein.

DHA can be present in an amount of 50 mg to 150 mg per serving, or any range or subrange between 50 mg and 150 mg.

The large breed puppy companion supplement can comprise amino acids. The amino acids comprises at least one of a L-Carnitine, a tryptophan, or any combination thereof.

Tryptophan can be present in an amount of 125 mg to 175 mg per serving, or any range or subrange between 125 mg and 175 mg.

L-Carnitine can be present in the large breed puppy companion supplement in a precise dose. The L-Carnitine can be present in an amount of 100 mg per serving.

The companion supplement can comprise a joint support blend. The joint support blend comprises a glucosamine HCl, a chondroitin sulfate, a methylsulfonylmethane (MSM), collagen peptides, a manganese, or any combination thereof.

Glucosamine HCl can be present in the large breed puppy companion supplement in a precise ratio for optimal absorption compared to sulfate forms. Glucosamine HCl can be present in an amount of 175 mg to 225 mg per serving, or any range or subrange between 175 mg and 225 mg.

MSM can be present in the companion supplement in a precise dose. The MSM can be present in an amount of 150 mg per serving. Chondroitin sulfate can be present in the companion supplement in a precise ratio for optimal absorption. Chondroitin sulfate can be present in an amount of 100 mg to 200 mg per serving, or any range or subrange between 125 mg and 175 mg.

Collagen peptides can be present in an amount of 50 mg to 150 mg per serving, or any range or subrange between 50 mg and 150 mg.

Manganese can be present in an amount of 1 mg to 10 mg per serving, or any range or subrange between 1 mg and 10 mg.

The large breed puppy companion supplement can comprise a digestive support blend. The digestive support blend can comprise at least one of an inulin, a fructooligosaccharides, a mannanoligosaccharides, Beta-glucans, arabinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend. The prebiotic blend can comprise the inulin, the fructooligosaccharides the mannanoligosaccharides, the Beta-glucans, and the arabinogalactans.

Inulin can be present in an amount of 50 mg to 150 mg per serving, or any range or subrange between 50 mg and 150 mg.

Fructooligosaccharides can be present in an amount of 50 mg to 100 mg per serving, or any range or subrange between 50 mg and 100 mg.

Mannanoligosaccharides can be present in an amount of 25 mg to 75 mg per serving, or any range or subrange between 25 mg and 75 mg.

Beta-glucans can provide immune-modulating prebiotics. Beta-glucans can be present in an amount of 15 mg to 35 mg per serving as disclosed herein.

Arabinogalactans can provide a plant-based prebiotic to support bacteria growth. Arabinogalactans can be present in an amount of 15 mg to 35 mg per serving, or any range or subrange between 15 mg and 35 mg.

Bromelain is a protein-digesting enzyme. Bromelain can be present in an amount of 15 mg to 35 mg per serving, or any range or subrange between 10 mg and 30 mg.

Ginger root extract can provide support for digestive comfort and motility. Ginger root extract can be present in an amount of 15 mg to 35 mg per serving, or any range or subrange between 15 mg and 35 mg.

The large breed puppy companion supplement can comprise a probiotic blend. For example, in some embodiments, the probiotic blend comprises at least one of lactobacillus, bifidobacterium, Enterococcus faecium, Streptococcus thermophilus, or any combination thereof.

The probiotic blend can be present in an amount of 2 billion colony-forming units (CFU) to 4 billion CFU per serving, or any range or subrange between 2 billion CFU to 4 billion CFU. For example, in some embodiments, the probiotic blend can be present in an amount of 2.5 billion CFU to 3.5 billion CFU, 2 billion CFU to 3.5 billion CFU, 2 billion CFU to 3 billion CFU, 2 billion CFU to 2.5 billion CFU, 2.5 billion CFU to 4 billion CFU, 3 billion CFU to 4 billion CFU, or 3.5 billion CFU to 4 billion CFU per serving.

The large breed puppy companion supplement can comprise a herbal blend. The herbal blend can comprise at least one of a chamomile extract, a fennel seed extract, a boswellia, or any combination thereof.

The chamomile extract can be present in an amount of 30 mg to 70 mg per serving, or any range or subrange between 30 mg and 70 mg.

The fennel seed extract can be present in an amount of 15 mg to 35 mg per serving, or any range or subrange between 15 mg and 35 mg.

The boswellia can comprise 65% boswellic acids. The boswellia can be present in an amount of 10 mg to 50 mg per serving, or any range or subrange between 10 mg and 50 mg. For example, in some embodiments, boswellia can be present in an amount of 15 mg to 45 mg, 20 mg to 40 mg, or 25 mg to 35 mg per serving. In some embodiments, boswellia can be present in an amount of 15 mg to 50 mg, 20 mg to 50 mg, 25 mg to 50 mg, 30 mg to 50 mg, 35 mg to 50 mg, 40 mg to 50 mg, or 45 mg to 50 mg per serving. In some embodiments, boswellia can be present in an amount of 10 mg to 45 mg, 10 mg to 40 mg, 10 mg to 35 mg, 10 mg to 30 mg, 10 mg to 25 mg, 10 mg to 20 mg, or 10 mg to 15 mg per serving.

The large puppy breed companion supplement can comprise a growth support blend. The herbal blend can comprise at least one of a colostrum, nucleotides, egg protein peptides, choline, or any combination thereof.

The colostrum can be present in an amount of 50 mg to 100 mg per serving, or any range or subrange between 50 mg to 150 mg.

The nucleotides can be present in an amount of 50 mg to 100 mg per serving, or any range or subrange between 50 mg to 100 mg.

The egg protein peptides can be present in an amount of 50 mg to 100 mg per serving, or any range or subrange between 50 mg to 100 mg.

The choline can be present in an amount of 25 mg to 75 mg per serving, or any range or subrange between 25 mg to 75 mg.

The large breed puppy companion supplement can comprise a Coenzyme Q10. Coenzyme Q10 can be present in the companion supplement in a precise dose. The Coenzyme Q10 can be present in an amount of 15 mg per serving.

The small/medium breed companion supplement can be administered simultaneously (“co-administered”) with administering the small/medium breed KibbleX composition. In some embodiments, the companion supplement can be co-administered about 1 second to 5 minutes of administering the small/medium breed KibbleX composition, or any range or subrange between about 1 second and 5 minutes as disclosed herein. For example, in some embodiments, the small/medium breed companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the kibble composition.

Table 9 is an exemplary illustration of ingredients in an embodiment of a KibbleX Large Breed Puppy Companion Supplement of the present disclosure developed in accordance with a method of the present disclosure based on the KibbleX Large Breed Puppy base food having the guaranteed analysis nutritional information listed in Table 10.

TABLE 9
Supplement Facts
Serving Size 1 Soft Chew (4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 35 | IU | † |
| tocopherols) | | | |
| Minerals | | | |
| Zinc (as Zinc Carnosine) | 15 | mg | † |
| DHA (from Algae) | 100 | mg | † |
| Biotin | 250 | mcg | † |
| Amino Acids | | | |
| L-Carnitine | 100 | mg | † |
| Tryptophan | 150 | mg | † |
| Joint Development Blend | 605 | mg | |
| Glucosamine HCl | 200 | mg | † |
| Chondroitin Sulfate | 150 | mg | † |
| MSM | 150 | mg | † |
| Collagen Peptides | 100 | mg | † |
| Manganese | 5 | mg | † |
| Digestive Support Blend | 275 | mg | |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 25 | mg | † |
| Bromelain | 25 | mg | † |
| Lipase | TBD | | † |
| Protease | TBD | | † |
| Amylase | TBD | | |
| Probiotic Blend | 3 billion CFU | | † |
| Growth Support Blend | 275 | mg | |
| Colostrum | 75 | mg | † |
| Nucleotides | 75 | mg | † |
| Egg Protein Peptides | 75 | mg | † |
| Choline | 50 | mg | † |
| Herbal Blend | 155 | mg | |
| Chamomile Extract | 50 | mg | † |
| Fennel Seed Extract | 25 | mg | † |
| Boswellia | 30 | mg | † |
| (65% boswellic acids) | | | |
| Other Nutrients | | | |
| Coenzyme Q10 | 15 | mg | † |

TABLE 10
Guaranteed Analysis - Large Breed Puppy Base Food

| Crude Protein (min) | 25.0% | |
|---|---|---|
| Crude Fat (min) | 14.0% | |
| Crude Fiber (max) | 4.5% | |
| Moisture (max) | 10.0% | |
| Docosahexaenoic Acid (DHA) (min) | 0.12% | |
| Eicosapentaenoic Acid (EPA) (min) | 0.14% | |
| Calcium (min) | 1.2% | |
| Phosphorus (min) | 1.0% | |
| Vitamin A (min) | 24,000 | IU/kg |
| Vitamin E (min) | 350 | IU/kg |
| Ascorbic Acid (Vitamin C) (min) | 140 | mg/kg |
| Omega-3 Fatty Acids (min) | 0.60% | |
| Omega-6 Fatty Acids (min) | 3.00% | |
| Taurine (min) | 0.20% | |
| Total microorganisms (min) | 10,000,000 | CFU/lb |

In some embodiments, a method comprises administering between about 2 ounces to 6 ounces of an adult weight loss KibbleX composition to a dog, or any range of subrange between 2 ounces to 6 ounces.

In some embodiments, the administering comprises pouring between about 2 ounces to 6 ounces of an adult weight loss KibbleX composition to a dog. In some embodiments, the administering comprises scooping between about 2 ounces to 6 ounces of an adult weight loss KibbleX composition to a dog. In some embodiments, the administering comprises dispensing between about 2 ounces to 6 ounces of an adult weight loss KibbleX composition to a dog. In some embodiments, the administering comprises between about 2 ounces to 6 ounces of an adult weight loss KibbleX composition to a dog. In some embodiments, the administering comprises distributing between about 2 ounces to 6 ounces of an adult weight loss KibbleX composition to a dog. In some embodiments, administering comprises ingesting between about 2 ounces to 6 ounces of an adult weight loss KibbleX composition to a dog.

The adult weight loss KibbleX composition can have an improved protein composition compared to traditional adult weight loss kibble compositions. The adult weight loss KibbleX composition can provide protein-centric composition. In some embodiments, the protein from a protein source comprises egg powder.

In some embodiments, the adult weight loss KibbleX composition can comprise about 20% to 35% protein from a protein source on a total weight basis of the adult weight loss KibbleX composition, or any range or subrange between about 20% and 35%. For example, in some embodiments, the adult weight loss KibbleX composition can comprise about 25% to 30%, 20% to 30%, 20% to 25%, 25% to 35%, or 30% to 35% protein from a protein source on a total weight basis of the adult weight loss KibbleX composition.

The adult weight loss KibbleX composition can comprise a low glycemic carbohydrates comprising at least one of brown rice, oat groats, pearled barley, sorghumor any combination thereof. Brown rice can be the primary carbohydrate source.

In some embodiments, the adult weight loss KibbleX composition can comprise about 30% to 55% grains on a total weight basis of the adult weight loss KibbleX composition, or any range or subrange between 30% and 55%. For example, in some embodiments, the adult weight loss KibbleX composition can comprise about 35% to 50%, 35% to 45%, 30% to 50%, 30% to 45%, 30% to 40%, 30% to 35%, 35% to 55%, 40% to 55%, 45% to 55%, 50% to 55% grains on a total weight basis of the adult weight loss KibbleX composition.

In some embodiments, the adult weight loss KibbleX composition can comprise about 1% to 15% chicken fat on a total weight basis of the KibbleX composition, or any range or subrange between 1% and 15%. For example, in some embodiments, the adult weight loss KibbleX composition can comprise about 5% to 10%, 1% to 10%, 1% to 5%, 5% to 15%, or 5% to 10% chicken fat on a total weight basis of the adult weight loss KibbleX composition.

The adult weight loss KibbleX composition can have a higher protein content than an KibbleX All Life Stage composition to support lean muscle mass. The adult weight loss KibbleX composition can have a reduced caloric density. For example, the caloric density can be 3,439 kcal/kg (330 kcal/cup) compared to 3,706 kcal/kg (458 kcal/cup) in the KibbleX All Life Stage composition.

The adult weight loss KibbleX composition provides a proprietary fiber and prebiotic mix. In some embodiments, fiber sources can comprise at least one of a beet pulp, a rice brain, an oat groat, a pearled barley, or any combination thereof.

In some embodiments, the method comprises administering between about 3 to 5 grams of an adult weight loss companion supplement. In some embodiments, the administering comprises pouring between about 3 to 5 grams of an adult weight loss companion supplement. In some embodiments, the administering comprises scooping between about 3 to 5 grams of an adult weight loss companion supplement. In some embodiments, the administering comprises dispensing between about 3 to 5 grams of an adult weight loss companion supplement. In some embodiments, the administering comprises between about 3 to 5 grams of an adult weight loss companion supplement. In some embodiments, the administering comprises distributing between about 3 to 5 grams of an adult weight loss companion supplement. In some embodiments, administering comprises ingesting between about 3 to 5 grams of an adult weight loss companion supplement.

The adult weight loss companion supplement can be effective weight management and healthy weight loss for dogs.

The adult weight loss companion supplement can be tailored to complement the adult weight loss KibbleX composition. For example, the adult weight loss companion supplement can be a KibbleX Adult Weight Loss Companion Supplement. The adult weight loss companion supplement can be in the form of a soft chew.

The adult weight loss companion supplement can optimize protein utilization, provide joint support, provide digestive support, improve skin and coat health, enhanced fat metabolism, optimize protein utilization, provide joint support during weight loss, provide comprehensive digestive support, improve skin and coat health, provide blood sugar management, provide enhance satiety support in synergy with the adult weight loss kibble composition.

The adult weight loss companion supplement can optimize protein utilization. It will be appreciated that any one or more of vitamins, minerals, amino acids, amino acid derivatives, fats, herbs, probiotics, proteins, carbohydrates, complex carbohydrates, prebiotic fibers, soluble fibers, enzymes, proteolytic enzymes, extracts, bioactive compounds, among other ingredients disclosed herein can comprise the adult weight loss companion supplement. It will further be appreciated that adult weight loss companion supplement can comprise the ingredients present in an amount similar, or the same as the companion supplement disclosed herein.

The adult weight loss companion supplement can deliver heat-sensitive nutrients at the nutrient's effective potency as disclosed herein. The adult weight loss companion supplement can have precise dosing of the nutrients to ensure optimal nutrient levels.

The adult weight loss companion supplement can comprise at least one of Vitamin C, a Vitamin E, or any combination thereof. The Vitamin C can be present in an amount of 30 mg to 70 mg per serving, or any range or subrange between 30 milligrams mg and 70 mg as disclosed herein.

The Vitamin E can be present in an amount of 20 IU to 60 IU per serving, or any range or subrange between 20 IU and 60 IU.

The adult weight loss companion supplement can comprise a skin and coat blend. The skin and coat blend comprises at least one of a zinc, DHA, a biotin, or any combination thereof. The adult weight loss companion supplement can elevate skin and coat support without requiring precise dosing of various nutrients.

Zinc can be present in the adult weight loss companion supplement in a precise dose. The zinc can be present in an amount of 20 mg per serving.

Biotin can be present in an amount of 200 micrograms (mcg) to 300 mcg per serving, or any range or subrange between 200 mcg and 300 mcg as disclosed herein.

Omega-3 Fatty Acids can be present in an amount of 100 mg to 200 mg per serving, or any range or subrange between 100 mg and 200 mg. In some embodiments, the Omega-3 Fatty Acids can be present in an amount of 110 mg to 190 mg, 120 mg to 180 mg, 130 mg to 170 mg, or 140 mg to 160 mg per serving. In some embodiments, the Omega-3 Fatty Acids can be present in an amount of 110 mg to 200 mg, 120 mg to 200 mg, 130 mg to 200 mg, 140 mg to 200 mg, 150 mg to 200 mg, 160 mg to 200 mg, 170 mg to 200 mg, 180 mg to 200 mg, or 190 mg to 200 mg per serving.

The adult weight loss companion supplement can comprise amino acids. The amino acids comprises at least one of a L-Carnitine, a tryptophan, or any combination thereof.

Tryptophan can be present in an amount of 10 mg to 50 mg per serving, or any range or subrange between 10 mg and 50 mg. For example, in some embodiments, tryptophan can be present in an amount of 15 mg to 45 mg, 20 mg to 40 mg, or 25 mg to 35 mg per serving. In some embodiments, tryptophan can be present in an amount of 15 mg to 50 mg, 20 mg to 50 mg, 25 mg to 50 mg, 30 mg to 50 mg, 35 mg to 50 mg, 40 mg to 50 mg, or 45 mg to 50 mg per serving. In some embodiments, tryptophan can be present in an amount of 10 mg to 45 mg, 10 mg to 40 mg, 10 mg to 35 mg, 10 mg to 30 mg, 10 mg to 25 mg, 10 mg to 20 mg, or 10 mg to 15 mg per serving.

L-Carnitine can be present in the adult weight loss companion supplement in a precise dose. The L-Carnitine can be present in an amount of 250 mg per serving.

The adult weight loss companion supplement can comprise a joint support blend. The joint support blend comprises a glucosamine HCl, a chondroitin sulfate, a MSM, collagen peptides, a manganese, or any combination thereof.

Glucosamine HCl can be present in an amount of 250 mg to 300 mg per serving, or any range or subrange between 250 mg and 300 mg as disclosed herein.

MSM can be present in the lamb meal and brown rice companion supplement in a precise dose. The MSM can be present in an amount of 175 mg per serving.

Chondroitin sulfate can be present in an amount of 150 mg to 200 mg per serving, or any range or subrange between 150 mg and 200 mg as disclosed herein.

Collagen peptides can be present in an amount of 100 mg to 200 mg per serving, or any range or subrange between 100 mg and 200 mg as disclosed herein.

Manganese can be present in an amount of 1 mg to 10 mg per serving, or any range or subrange between 1 mg and 10 mg.

The adult weight loss companion supplement can comprise a digestive support blend. The digestive support blend can comprise at least one of an inulin, a fructooligosaccharides, a mannanoligosaccharides, Beta-glucans, arabinogalactans, a ginger root extract, a bromelain, a lipase, a protease, an amylase, or any combination thereof.

The digestive support blend can comprise a prebiotic blend to support gut health, microbiome balance, and nutrient absorption. In some embodiments, the prebiotic blend is Firmtastic®, a proprietary fiber blend.

Inulin can be present in an amount of 50 mg to 150 mg per serving, or any range or subrange between 50 mg and 150 mg as disclosed herein.

Fructooligosaccharides can be present in an amount of 50 mg to 100 mg per serving, or any range or subrange between 50 mg and 100 mg as disclosed herein.

Mannanoligosaccharides can be present in an amount of 25 mg to 75 mg per serving, or any range or subrange between 25 mg and 75 mg as disclosed herein.

Beta-glucans can be present in an amount of 15 mg to 35 mg per serving, or any range or subrange between 15 mg and 35 mg as disclosed herein.

Arabinogalactans can be present in an amount of 15 mg to 35 mg per serving, or any range or subrange between 15 mg and 35 mg as disclosed herein.

Bromelain can be present in an amount of 10 mg to 30 mg per serving, or any range or subrange between 10 mg and 30 mg. For example, in some embodiments, bromelain can be present in an amount of 15 mg to 25 mg, 15 mg to 30 mg, 20 mg to 30 mg, 25 mg to 30 mg, 10 mg to 25 mg, 10 mg to 20 mg, or 10 mg to 15 mg per serving.

Ginger root extract can provide support for digestive comfort and motility. Ginger root extract can be present in an amount of 15 mg to 35 mg per serving, or any range or subrange between 15 mg and 35 mg.

The adult weight loss companion supplement can comprise a probiotic blend. For example, in some embodiments, the probiotic blend comprises at least one of lactobacillus, bifidobacterium, Enterococcus faecium, Streptococcus thermophilus, or any combination thereof.

The probiotic blend can be present in an amount of 1 billion CFU to 2 billion CFU per serving, or any range or subrange between 1 billion CFU to 2 billion CFU as disclosed herein.

The adult weight loss companion supplement can comprise a herbal blend. The herbal blend can comprise at least one of a siliphos, an ashwagandha KSM-66, a kelp powder, or any combination thereof.

The siliphos can be present in an amount of 25 mg to 75 mg per serving, or any range or subrange between 25 mg to 75 mg.

The ashwagandha KSM-66 can be present in an amount of 25 mg to 50 mg per serving, or any range or subrange between 25 mg to 50 mg.

The kelp powder can be present in an amount of 10 mg to 30 mg per serving, or any range or subrange between 10 mg to 30 mg.

The adult weight loss companion supplement can comprise a metabolic support blend. The metabolic support blend can comprise a green tea extract, conjugated linoleic acid (CLA), a chromium, a cinnamon bark extract, or any combination thereof.

The green tea extract can comprise decaffeinated, standardized to 45% EGCG. The green tea extract can be present in an amount of 25 mg to 75 mg per serving, or any range or subrange between 25 mg to 75 mg. For example, in some embodiments, green tea extract can be present in an amount of 30 mg to 70 mg, 35 mg to 65 mg, 40 mg to 60 mg, or 45 mg to 55 mg per serving. In some embodiments, green tea extract can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, green tea extract can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving.

The CLA can be present in an amount of 50 mg to 100 mg per serving, or any range or subrange between 50 mg to 100 mg. For example, in some embodiments, CLA can be present in an amount of 55 mg to 95 mg, 60 mg to 90 mg, 65 mg to 85 mg, or 70 mg to 80 mg per serving. In some embodiments, CLA can be present in an amount of 55 mg to 100 mg, 60 mg to 100 mg, 65 mg to 100 mg, 70 mg to 100 mg, 75 mg to 100 mg, 80 mg to 100 mg, 85 mg to 100 mg, 90 mg to 100 mg, or 95 mg to 100 mg per serving. In some embodiments, CLA can be present in an amount of 50 mg to 95 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg per serving.

The chromium can comprise chromium picolinate. The chromium can be present in an amount of 25 mg to 75 mg per serving, or any range or subrange between 25 mg to 75 mg. For example, in some embodiments, chromium can be present in an amount of 30 mg to 70 mg, 35 mg to 65 mg, 40 mg to 60 mg, or 45 mg to 55 mg per serving. In some embodiments, chromium can be present in an amount of 30 mg to 75 mg, 35 mg to 75 mg, 40 mg to 75 mg, 45 mg to 75 mg, 50 mg to 75 mg, 55 mg to 75 mg, 60 mg to 75 mg, 65 mg to 75 mg, or 70 mg to 75 mg per serving. In some embodiments, chromium can be present in an amount of 25 mg to 70 mg, 25 mg to 65 mg, 25 mg to 60 mg, 25 mg to 55 mg, 25 mg to 50 mg, 25 mg to 45 mg, 25 mg to 40 mg, 25 mg to 35 mg, or 25 mg to 30 mg per serving.

Cinnamon bar extract can be present in an amount of 15 mg to 35 mg per serving, or any range or subrange between 15 mg and 35 mg. For example, in some embodiments, cinnamon bar extract can be present in an amount of 20 mg to 30 mg, 15 mg to 30 mg, 15 mg to 25 mg, 15 mg to 20 mg, 20 mg to 35 mg, 25 mg to 35 mg, or 30 mg to 35 per serving.

The adult weight loss companion supplement can comprise at least one of a choline, a Coenzyme Q10, or any combination thereof. Coenzyme Q10 can be present in the companion supplement in a precise dose. The Coenzyme Q10 can be present in an amount of 15 mg per serving.

The choline can be present in an amount of 25 mg to 75 mg per serving, or any range or subrange between 25 mg to 75 mg.

The adult weight loss supplement can be administered simultaneously (“co-administered”) with administering the adult weight loss KibbleX composition. In some embodiments, the companion supplement can be co-administered about 1 second to 5 minutes of administering the adult weight loss KibbleX composition, or any range or subrange between about 1 second and 5 minutes as disclosed herein. For example, in some embodiments, the adult weight loss companion supplement can be co-administered about 30 seconds to 4 minutes, 1 minute to 3 minutes, 1 second to 4 minutes, 1 second to 3 minutes, 1 second to 2 minutes, 1 second to 1 minute, 30 seconds to 5 minutes, 1 minute to 5 minutes, 2 minutes to 5 minutes, 3 minutes to 5 minutes, or 4 minutes to 5 minutes of administering the kibble composition.

Table 11 is an exemplary illustration of ingredients in an embodiment of a KibbleX Adult Weight Loss Companion Supplement of the present disclosure developed in accordance with a method of the present disclosure based on the KibbleX Adult Weight Loss base food having the guaranteed analysis nutritional information listed in Table 12.

TABLE 11
Supplement Facts
Serving Size 1 Soft Chew (4 g) Servings Per Container 60

| Nutrient | Amount Per Serving | | % Daily Value* |
|---|---|---|---|
| Vitamins | | | |
| Vitamin C (as Ester-C) | 50 | mg | † |
| Vitamin E (as mixed | 30 | IU | † |
| tocopherols) | | | |
| Skin & Coat Blend | | | |
| Zinc (as Zinc Carnosine) | 20 | mg | † |
| Omega-3 Fatty Acids | 150 | mg | † |
| (from Krill Oil) | | | |
| Biotin | 250 | mcg | † |
| Amino Acids | 380 | mg | |
| L-Carnitine | 250 | mg | † |
| L-Glutamine | 100 | mg | † |
| Tryptophan | 30 | mg | † |
| Joint Support Blend | 775 | mg | |
| Glucosamine HCl | 275 | mg | † |
| Chondroitin Sulfate | 175 | mg | † |
| MSM (Methylsulfonylmethane) | 175 | mg | † |
| Collagen Peptides | 150 | mg | † |
| Manganese | 5 | mg | † |
| Digestive Support Blend | 321 | mg | |
| Inulin | 100 | mg | † |
| Fructooligosaccharides (FOS) | 75 | mg | † |
| Mannanoligosaccharides (MOS) | 50 | mg | † |
| Beta-glucans | 25 | mg | † |
| Arabinogalactans | 25 | mg | † |
| Ginger Root Extract | 25 | mg | † |
| Bromelain | 20 | mg | † |
| Lipase | TBD | | † |
| Protease | TBD | | † |
| Amylase | TBD | | |
| Probiotic Blend | 2 billion CFU | | † |
| Metabolic Support Blend | 150 | mg | |
| Green Tea Extract | 50 | mg | † |
| (decaffeinated, | | | |
| standardized | | | |
| to 45% EGCG) | | | |
| CLA (Conjugated | 75 | mg | † |
| Linoleic Acid) | | | |
| Chromium (as | 50 | mcg | † |
| Chromium Picolinate) | | | |
| Cinnamon Bark Extract | 25 | mg | † |
| Herbal Blend | 105 | mg | |
| Siliphos | 50 | mg | † |
| Ashwagandha KSM-66 | 35 | mg | † |
| Kelp Powder | 20 | mg | † |
| Other Nutrients | | | |
| Coenzyme Q10 | 15 | mg | † |

TABLE 12
Guaranteed Analysis - Adult Weight Loss Base Food

| Crude Protein (min) | 30.0% | |
|---|---|---|
| Crude Fat (min) | 11.5% | |
| Crude Fiber (max) | 5.0% | |
| Moisture (max) | 10.0% | |
| Docosahexaenoic Acid (DHA) (min) | 0.12% | |
| Eicosapentaenoic Acid (EPA) (min) | 0.14% | |
| Calcium (min) | 1.4% | |
| Phosphorus (min) | 1.0% | |
| Vitamin A (min) | 24,000 | IU/kg |
| Vitamin E (min) | 500 | IU/kg |
| Ascorbic Acid (Vitamin C) (min) | 200 | mg/kg |
| Omega-3 Fatty Acids (min) | 0.60% | |
| Omega-6 Fatty Acids (min) | 2.60% | |
| Taurine (min) | 0.12% | |
| L-Carnitine (min) | 50 | mg/kg |
| Total microorganisms (min) | 10,000,000 | CFU/lb |

Any one or more of the embodiments disclosed herein shall be understood to be combinable without departing from the scope or spirit of the disclosure. Aspect 1. A method for a well-rounded nutritional plan for a dog comprising: administering between about 2 ounces to 6 ounces of a kibble composition to a dog, wherein the kibble composition comprises: about 20% to 35% protein from a protein source on a total weight basis of the KibbleX composition, wherein the protein source comprises at least one of a chicken meal, a turkey meal, lamb meal, or any combination thereof; about 30% to 55% grains on a total weight basis of the KibbleX composition; and about 5% to 20% chicken fat on a total weight basis of the KibbleX composition; and administering between about 3 to 5 grams of a grain companion supplement comprising: vitamins comprising at least one of Vitamin C, a Vitamin E, or any combination thereof; a skin and coat blend comprising at least one of a zinc, a biotin, an Omega-3 fatty acids, or any combination thereof; amino acids comprising at least one of a L-Carnitine, a tryptophan, or any combination thereof; a joint support blend comprising a glucosamine HCl, a chondroitin sulfate, a methylsulfonylmethane (MSM), collagen peptides, a manganese, or any combination thereof; a digestive support blend comprising at least one of an inulin, a fructooligosaccharides a mannanoligosaccharides Beta-glucans, arabinogalactans, a ginger root extract, a bromelain, a lipase, a protease, an amylase, or any combination thereof; a probiotic blend; a herbal blend comprising at least one of a siliphos, a curcumin, an ashwagandha KSM-66, a kelp powder, or any combination thereof; and at least one of a choline, a Coenzyme Q10, or any combination thereof.

Aspect 2. The method according to Aspect 1, wherein the grain companion supplement is in a form of a soft chew.

Aspect 3. The method according to any one of Aspects 1-2, wherein the grain companion supplement is administered between about 1 second to 5 minutes of administering the KibbleX composition.

Aspect 4. The method of claim 1, according to any one of Aspects 1-3, wherein the zinc is present in an amount of 20 mg per serving; wherein the Omega-3 fatty acids is present in an amount of 250 mg per serving; wherein the L-Carnitine is present in an amount of 100 mg per serving; and wherein the tryptophan is present in an amount of 125 mg per serving.

Aspect 5. The method according to any one of Aspects 1-4, wherein the Vitamin C is present in an amount of 30 mg to 70 mg per serving.

Aspect 6. The method according to any one of Aspects 1-5, wherein the Vitamin E is present in an amount of 10 IU to 50 IU per serving.

Aspect 7. The method according to any one of Aspects 1-6, wherein the L-Carnitine is present in an amount of 100 mg per serving.

Aspect 8. The method according to any one of Aspects 1-7, wherein the MSM is present in an amount of 175 mg per serving.

Aspect 9. The method according to any one of Aspects 1-8, wherein the Coenzyme Q10 is present in an amount of 15 mg per serving.

Aspect 10. The method according to any one of Aspects 1-9, wherein the Omega-3 fatty acids is present in an amount of 200 mg to 300 mg per serving.

Aspect 11. A method for a well-rounded nutritional plan for a dog comprising: administering between about 2 ounces to 6 ounces of a grain-free kibble composition to a dog, wherein the grain-free kibble composition comprises: about 35% to 55% protein from a protein source on a total weight basis of the grain-free KibbleX composition, wherein the protein source comprises at least one of a chicken meal, a turkey meal, peas, or any combination thereof; about 15% to 40% of a carbohydrate source on a total weight basis of the grain-free KibbleX composition, wherein the carbohydrate source comprising at least one of white potatoes, sweet potatoes, chickpeas, or any combination thereof; and about 5% to 20% chicken fat on a total weight basis of the grain-free KibbleX composition; and administering between about 3 to 5 grams of a grain-free companion supplement comprising: vitamins comprising at least one of Vitamin C, a Vitamin E, or any combination thereof; a skin and coat blend comprising at least one of a zinc, an Omega-3 fatty acids, a biotin, or any combination thereof; amino acids comprising at least one of a L-Carnitine, a tryptophan, or any combination thereof; a joint support blend comprising a glucosamine HCl, a chondroitin sulfate, a methylsulfonylmethane (MSM), collagen peptides, a manganese, or any combination thereof; a digestive support blend comprising at least one of an inulin, a fructooligosaccharides a mannanoligosaccharides Beta-glucans, arabinogalactans, a ginger root extract, a bromelain, a lipase, a protease, an amylase, or any combination thereof; a probiotic blend; a herbal blend comprising at least one of a siliphos, a curcumin, an ashwagandha KSM-66, a kelp powder, or any combination thereof; and at least one of a choline, a Coenzyme Q10, or any combination thereof.

Aspect 12. The method according to Aspect 11, wherein the grain-free companion supplement is in a form of a soft chew.

Aspect 13. The method according to any one of Aspects 11-12, wherein the grain-free companion supplement is administered between about 1 second to 5 minutes of administering the grain-free KibbleX composition.

Aspect 14. The method according to any one of Aspects 11-13, wherein the zinc is present in an amount of 15 mg per serving; wherein the Omega-3 fatty acids is present in an amount of 200 mg per serving; wherein the L-Carnitine is present in an amount of 150 mg per serving; and wherein the tryptophan is present in an amount of 70 mg per serving.

Aspect 15. The method according to any one of Aspects 11-14, wherein the Vitamin C is present in an amount of 30 mg to 70 mg per serving.

Aspect 16. The method according to any one of Aspects 11-15, wherein the Vitamin E is present in an amount of 10 IU to 50 IU per serving.

Aspect 17. The method according to any one of Aspects 11-16, wherein the L-Carnitine is present in an amount of 150 mg per serving.

Aspect 18. The method according to any one of Aspects 11-17, wherein the MSM is present in an amount of 70 mg per serving.

Aspect 19. The method according to any one of Aspects 11-18, wherein the tryptophan is present in an amount of 15 mg per serving.

Aspect 20. The method according to any one of Aspects 11-19, wherein the Omega-3 fatty acids is present in an amount of 200 mg per serving.

Aspect 21. A method of developing a companion supplement tailored to a dog's stage of life and nutritional requirements, the method comprising: choosing a base food format, wherein the base food format comprises base food ingredients; determining post-cook characteristics of the base food format, wherein the post-cook characteristics are selected from the group consisting of nutrient levels, temporal nutrient degradation modeling across an expected shelf-life of the base food format, bioavailability, digestibility, nutrient antagonisms, chemical form, oxidation losses over a shelf-life of the base food format, stability over a shelf-life of the base food format, and breed-specific risk factors, and combinations thereof; preparing a companion supplement based, at least in part, on the post-cook characteristics of the base food format, wherein the companion supplement comprises companion supplement ingredients, and wherein the companion supplement ingredients are selected from one of a limiting amino acid that is not present in the base food format, one or more enzymes calibrated to a protein:fat:carbohydrate ratio of the base food format, and combinations thereof; determining post-production characteristics of the companion supplement; and combining nutrient values of the base food format and the companion supplement to ensure safety and efficacy, and wherein, the companion supplement and the base food format, when administered together, are sufficiently designed to provide the dog with a nutritionally optimal feeding system for the dog based on the dog's stage of life and nutritional requirements.

Aspect 22. The method according to Aspect 21, wherein the base food format is grain-inclusive.

Aspect 23. The method according to Aspect 21, wherein the base food format is grain-free.

Aspect 24. The method according to any one of Aspects 21-23, wherein the base food format is selected from non-pressure agglomeration pellets, extruded pellets, freeze-dried food, air-dried food, cold-pressed food, or raw frozen food.

Aspect 25. The method according to any one of Aspects 21-24, wherein the base food format is for a dog selected from one of a puppy dog, an adult dog, a dog that needs to lose weight, and a pregnant dog.

Aspect 26. The method according to any one of Aspects 21-25 wherein the base food ingredients are selected from the group consisting of protein from a protein source, chicken fat, vitamins, and minerals, and combinations thereof.

Aspect 27. The method according to any one of Aspects 21-26, wherein the companion supplement ingredients comprise heat-sensitive vitamins that are different than any vitamins that are present in the base food ingredients.

Aspect 28. The method according to any one of Aspects 21-27, wherein determining characteristics of the base food format is determined after moisture is removed from the base food format.

Aspect 29. The method according to any one of Aspects 21-28, wherein the nutrient levels of the base food ingredients are characterized within about one week to about four weeks after manufacturing.

Aspect 30. The method according to any one of Aspect 21-29, wherein the nutrient levels of the base food ingredients are selected from one of vitamins, minerals, and amino acids, and combinations thereof.

Aspect 31. The method according to any one of Aspects 21-30, wherein the companion supplement ingredients are selected from one or more ingredients that have been shown to affect skin and coat health, joint support, digestive support, immune support, cognitive development, visual development, stress reduction, microbiome balance, and mental wellbeing, and combinations thereof.

Aspect 32. The method according to any one of Aspect 21-31, wherein the companion supplement is a soft chew.

Aspect 33. The method according to any one of Aspect 21-31, wherein the companion supplement is freeze-dried.

Aspect 34. A method for developing and producing a companion supplement tailored to a dog's stage of life and nutritional requirements for co-administration with a base food format, the method comprising: selecting a base food format comprising base food ingredients; manufacturing a representative commercial production batch under a standard production process for that format; determining a post-cook characterization set for the representative commercial production batch; identifying nutrients for a companion supplement based on at least one of: (a) sensitivity to thermal, pressure, and mechanical stresses during base food processing; (b) regulatory restrictions on inclusion in pet food; (c) functional benefits supporting optimal health beyond baseline nutritional adequacy; (d) a nutritional composition of the base food; and (e) any combinations thereof; developing the companion supplement for co-administration with the base food format, wherein the companion supplement comprises companion supplement ingredients selected to provide the dog with essential nutrients to ensure adequate intake of essential nutrients when co-administered, and wherein the companion supplement ingredients are selected from one of a limiting amino acid that is not present in the base food format, one or more enzymes calibrated to a protein:fat:carbohydrate ratio of the base food format, and combinations thereof; producing the companion supplement according to the developed companion supplement and packaging the companion supplement physically separate from the base food format; and providing administration instructions and labeling that identify compatibility of the packaged companion supplement with the corresponding base food format and specify co-administration at feeding, wherein the companion supplement is developed from the determined post-cook characterization set of the representative commercial production batch without reformulating the base food format, and when co-administered with the base food format, the companion supplement and the base food format are sufficiently designed to provide the dog with a nutritionally optimal feeding system based on the dog's stage of life and nutritional requirements.

Aspect 35. The method according to Aspect 34, wherein a base food format is grain-inclusive.

Aspect 36. The method according to Aspect 34, wherein a base food format is grain-free.

Aspect 37. The method according to any of Aspects 34-36, wherein the base food format is selected from non-pressure agglomeration pellets, extruded pellets, freeze-dried food, air-dried food, cold-pressed food, or raw frozen food.

Aspect 38. The method according to any of Aspects 34-37, wherein a base food format is for a dog selected from one of a puppy dog, an adult dog, a dog that needs to lose weight, and a pregnant dog.

Aspect 39. The method according to any of Aspects 34-38, wherein base food ingredients are selected from the group consisting of protein from a protein source, chicken fat, vitamins, and minerals, and combinations thereof.

Aspect 40. The method according to any of Aspects 34-39, wherein the companion supplement ingredients comprise heat-sensitive vitamins that are different than any vitamins that are present in the base food ingredients.

Aspect 41. The method according to any of Aspects 34-40 wherein determining a characterization set for each representative commercial production batch is determined after moisture is removed from the commercial production batch.

Aspect 42. The method according to any of Aspects 34-41, wherein the nutrient levels are characterized within about one week to about four weeks after manufacturing.

Aspect 43. The method according to any of Aspects 34-42, wherein the nutrient levels are selected from one of vitamins, minerals, and amino acids, and combinations thereof.

Aspect 44. The method according to any of Aspects 34-43, wherein the companion supplement ingredients are selected from one or more ingredients that have been shown to affect skin and coat health, joint support, digestive support, immune support, cognitive development, visual development, stress reduction, microbiome balance, and mental wellbeing, and combinations thereof.

Aspect 45. The method according to any of Aspects 34-44, wherein the companion supplement is a soft chew.

Aspect 46. The method according to any of Aspects 34-44, wherein the companion supplement is freeze-dried.

Aspect 47. The method according to any of Aspects 34-46, further comprising creating a base-format supplement mapping associating each base food format with a corresponding companion supplement formulation developed from its representative commercial production batch characterization, and providing labeling that identifies compatible base food formats for the packaged companion supplement.

Aspect 48. The method according to any of Aspects 34-47, further comprising verifying that, when co-administered according to the administration instructions, a net intake of nutrients meets stage-of-life requirements within predetermined tolerances for the base food format.

Aspect 49. The method according to any of Aspects 34,-48 wherein stability of the base food ingredients over a shelf-life is used to select antioxidant species and levels in the companion supplement to achieve a target shelf-life for the nutritionally optimal feeding system when co-administered.

Aspect 50. The method according to any of Aspects 34-49, wherein a chemical form of at least one companion supplement ingredient differs from the chemical form of the base food ingredients to increase predicted digestibility when co-administered.

What is claimed is:

1. A method of developing a companion supplement tailored to a dog's stage of life and nutritional requirements, the method comprising:
choosing a base food format,
wherein the base food format comprises base food ingredients;
determining post-cook characteristics of the base food format,
wherein the post-cook characteristics are selected from the group consisting of nutrient levels, temporal nutrient degradation modeling across an expected shelf-life of the base food format,
bioavailability, digestibility, nutrient antagonisms, chemical form, oxidation losses over a shelf-life of the base food format, stability over a shelf-life of the base food format, and breed-specific risk factors, and combinations thereof;
preparing a companion supplement based, at least in part, on the post-cook characteristics of the base food format,
wherein the companion supplement comprises companion supplement ingredients, and
wherein the companion supplement ingredients are selected from one of a limiting amino acid that is not present in the base food format, one or more enzymes calibrated to a protein:fat:carbohydrate ratio of the base food format, and combinations thereof, and
determining post-production characteristics of the companion supplement; and
combining nutrient values of the base food format and the companion supplement to ensure safety and efficacy, and
wherein, the companion supplement and the base food format, when administered together, are sufficiently designed to provide the dog with a nutritionally optimal feeding system for the dog based on the dog's stage of life and nutritional requirements.

2. The method of claim 1, wherein the base food format is grain-inclusive.

3. The method of claim 1, wherein the base food format is grain-free.

4. The method of claim 1, wherein the base food format is selected from non-pressure agglomeration pellets, extruded pellets, freeze-dried food, air-dried food, cold-pressed food, or raw frozen food.

5. The method of claim 1, wherein the base food format is for a dog selected from one of a puppy dog, an adult dog, a dog that needs to lose weight, and a pregnant dog.

6. The method of claim 1, wherein the base food ingredients are selected from the group consisting of protein from a protein source, chicken fat, vitamins, and minerals, and combinations thereof.

7. The method of claim 1, wherein the companion supplement ingredients comprise heat-sensitive vitamins that are different than any vitamins that are present in the base food ingredients.

8. The method of claim 1, wherein determining characteristics of the base food format is determined after moisture is removed from the base food format.

9. The method of claim 1, wherein the nutrient levels of the base food ingredients are characterized within about one week to about four weeks after manufacturing.

10. The method of claim 1, wherein the nutrient levels of the base food ingredients are selected from one of vitamins, minerals, and amino acids, and combinations thereof.

11. The method of claim 1, wherein the companion supplement ingredients are selected from one or more ingredients that have been shown to affect skin and coat health, joint support, digestive support, immune support, cognitive development, visual development, stress reduction, microbiome balance, and mental wellbeing, and combinations thereof.

12. The method of claim 1, wherein the companion supplement is a soft chew.

13. The method of claim 1, wherein the companion supplement is freeze-dried.

14. A method for developing and producing a companion supplement tailored to a dog's stage of life and nutritional requirements for co-administration with a base food format, the method comprising:
selecting a base food format comprising base food ingredients;
manufacturing a representative commercial production batch under a standard production process for that format;
determining a post-cook characterization set for the representative commercial production batch;
identifying nutrients for a companion supplement for the base food format based on at least one of: (a) sensitivity to thermal, pressure, and mechanical stresses during base food processing; (b) regulatory restrictions on inclusion in pet food; (c) functional benefits supporting optimal health beyond baseline nutritional adequacy; (d) a nutritional composition of the base food; and (e) any combinations thereof;
developing the companion supplement for co-administration with the base food format, wherein the companion supplement comprises companion supplement ingredients selected to provide the dog with essential nutrients to ensure adequate intake of essential nutrients when co-administered, and wherein the companion supplement ingredients are selected from one of a limiting amino acid that is not present in the base food format, one or more enzymes calibrated to a protein:fat:carbohydrate ratio of the base food format, and combinations thereof;
producing the companion supplement according to the developed companion supplement and packaging the companion supplement physically separate from the base food format; and
providing administration instructions and labeling that identify compatibility of the packaged companion supplement with the base food format and specify co-administration at feeding,
wherein the companion supplement is developed from the determined post-cook characterization set of the representative commercial production batch without reformulating the base food format, and when co-administered with the base food format, the companion supplement and the base food format are sufficiently designed to provide the dog with a nutritionally optimal feeding system based on the dog's stage of life and nutritional requirements.

15. The method of claim 14, wherein a base food format is grain-inclusive.

16. The method of claim 14, wherein a base food format is grain-free.

17. The method of claim 14, wherein the base food format is selected from non-pressure agglomeration pellets, extruded pellets, freeze-dried food, air-dried food, cold-pressed food, or raw frozen food.

18. The method of claim 14, wherein a base food format is for a dog selected from one of a puppy dog, an adult dog, a dog that needs to lose weight, and a pregnant dog.

19. The method of claim 14, wherein base food ingredients are selected from the group consisting of protein from a protein source, chicken fat, vitamins, and minerals, and combinations thereof.

20. The method of claim 14, wherein the companion supplement ingredients comprise heat-sensitive vitamins that are different than any vitamins that are present in the base food ingredients.

21. The method of claim 14 wherein determining a characterization set for each representative commercial production batch is determined after moisture is removed from the commercial production batch.

22. The method of claim 14, wherein the nutrient levels are characterized within about one week to about four weeks after manufacturing.

23. The method of claim 14, wherein the nutrient levels are selected from one of vitamins, minerals, and amino acids, and combinations thereof.

24. The method of claim 14, wherein the companion supplement ingredients are selected from one or more ingredients that have been shown to affect skin and coat health, joint support, digestive support, immune support, cognitive development, visual development, stress reduction, microbiome balance, and mental wellbeing, and combinations thereof.

25. The method of claim 14, wherein the companion supplement is a soft chew.

26. The method of claim 14, wherein the companion supplement is freeze-dried.

27. The method of claim 14, further comprising creating a base-format supplement mapping associating each base food format with a corresponding companion supplement formulation developed from its representative commercial production batch characterization, and providing labeling that identifies compatible base food formats for the packaged companion supplement.

28. The method of claim 14, further comprising verifying that, when co-administered according to the administration instructions, a net intake of nutrients meets stage-of-life requirements within predetermined tolerances for the base food format.

29. The method of claim 14, wherein stability of the base food ingredients over a shelf-life is used to select antioxidant species and levels in the companion supplement to achieve a target shelf-life for the nutritionally optimal feeding system when co-administered.

30. The method of claim 14, wherein a chemical form of at least one companion supplement ingredient differs from the chemical form of the base food ingredients to increase predicted digestibility when co-administered.
